(19) United States
(12) Patent Application Publication
(10) Pub. No.: US 2012/0052041 A1
(43) Pub. Date: Mar. 1, 2012

(54) POLYMERIC NANOPARTICLES WITH ENHANCED DRUG-LOADING AND METHODS OF USE THEREOF

(75) Inventors: Sudipta Basu, Cambridge, MA (US); Rania Harfouche, Allston, MA (US); Shivani Soni, Montgomery, AL (US); Shiladitya Sengupta, Waltham, MA (US)

(73) Assignee: THE BRIGHAM AND WOMEN'S HOSPITAL, INC., Boston, MA (US)

(21) Appl. No.: 13/147,755

(22) PCT Filed: Feb. 4, 2010
(86) PCT No.: PCT/US2010/023212

§ 371 Date: Nov. 16, 2011

Related U.S. Application Data
(60) Provisional application No. 61/149,779, filed on Feb. 4, 2009.

(51) Int. Cl. A61K 47/48; A61P 35/00; A61K 47/34; B82Y 5/00

(52) U.S. Cl. 424/78.17; 514/772.1; 514/772.2; 977/773; 977/788; 977/906

(57) ABSTRACT

The invention is directed to modified polymers with increased drug-loading including compounds of formula (I): wherein Z is a poly(lactic-co-glycolic acid) (PLGA) polymer having molecular weight from 1-15 kDa and where the ratio of lactide to glycolide in the PLGA polymer is from 1:10 to 10:1; formula (II) R₁ are independently H, R₂, OH, O-alkyl, —O—R₂, NH—R₂, -linker-R₂, or -and R₂ are independently one or more therapeutic agents. The invention is also directed to nanoparticle drug delivery systems including a PLGA-b-PEG block copolymer; and a stabilizer and to drug delivery systems including PLGA-b-PEG block copolymer polyvinyl alcohol (PVA) nanoparticle; and the modified polymer substantially as described herein.

### RELATED APPLICATIONS

This application claims priority to and benefit under 35 U.S.C. §119(e) of the U.S. Provisional Application Nos. 61/149,779, filed Feb. 4, 2009, the content of which is incorporated herein by reference its entirety.

### GOVERNMENT SUPPORT

The subject matter of this application was made with support Department of Defense Breast Cancer Research Program Era of Hope Scholar award W81XWH-07-1-0482. The U.S. Government has certain rights in this invention.

### FIELD OF THE INVENTION

This invention relates to modified polymers with increased drug-loading, nanoparticle drug delivery systems, and methods of use thereof.

### BACKGROUND OF THE INVENTION

Cancer is the second leading cause of mortality in the United States, with an estimated 1,444,180 new cases and 565,650 deaths in 2008 [1]. Cytotoxic agents, which are used in standard chemotherapy, non-specifically target all dividing cells resulting in dose-limiting toxicities. There is an urgent need to develop novel strategies that are more specifically targeted against the tumor.

The mitogen activated protein kinase (MAPK) pathway comprising of RAS, RAF, MEK and ERK has been implicated in most human tumors, often through gain of function mutations in RAS and RAF family [2-3]. Indeed, RAS mutations are found in 30% of all cancer, and are in particular common in pancreatic cancer (90%) [4], colon cancer (50%) [5], while RAF mutations are prevalent in melanomas (63%) [6] and ovarian cancer (36%) [7]. As a result the MAPK pathway has evolved as a focus of intense investigation for developing small molecule inhibitors as targeted therapeutics. Many of these small molecule inhibitors are currently in clinical trials and have shown target suppression and tumor inhibition in Phase I studies (4).

### SUMMARY OF THE INVENTION

The invention is directed to modified polymers with increased drug-loading including compounds of formula (I):

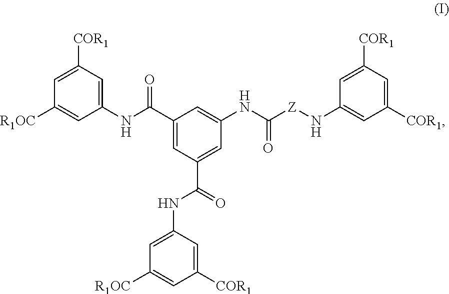

wherein Z is polymer having molecular weight from 1-15 kDa;
R₁ are independently H, R₂, OH, O-alkyl, —O—R₂, NH—R₂, -linker-R₂, or

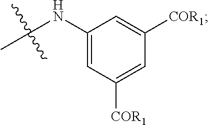

and
R₂ are independently one or more therapeutic agents.

Another aspect of the invention is directed to nanoparticle drug delivery systems including a PLGA-b-PEG block copolymer; and a stabilizer.

Yet another aspect of the invention is directed to drug delivery systems including PLGA-b-PEG block copolymer polyvinyl alcohol (PVA) nanoparticle; and the modified polymer substantially as described herein.

### BRIEF DESCRIPTION OF THE DRAWINGS

FIG. 1A is a schematic representation showing the synthesis of different PLGA-(PD98059)x conjugates.

FIG. 1B shows loading of PD98059 in mono-, tri- and hexa-conjugated PD98059-PLGA expressed as μg per mg of polymer.

FIG. 2A shows a synthetic scheme for PEG-b-PLGA conjugate for engineering pegylated nanoparticles. Different ratio of PLGA-PEG:PLGA-6[PD] results in nanoparticles of different size distribution as measured by dynamic light scattering (DLS).

FIG. 2B shows physicochemical release kinetics in different cell lysates (MDA-MB231, LLC and B16/F10), demonstrating sustained release of active PD98059 from nanoparticles. FIG. 2C shows a schematic representation of surface coating of nanoparticles with PEG. Bioitinylated nanoparticles were engineered from PLGA-b-PEG-biotin conjugate and probed with 5 nm streptavidine-gold NP. The nanoparticles were cross-sectioned and imaged using TEM. The TEM image of the cross section of a gold-NP coated pegylated nanoparticle showed that PLGA-PEG core with dark gold-NP at the surface (data not shown). The DLS graph shows the size distribution of the biotinylated-pegylated nanoparticles.

FIG. 3A shows bar graphs of results from MTS assays to determine the temporal cytotoxicity of increasing concentrations of free PD98059 and PD98059-nanoparticles (NP). Data represents mean±SEM (n=3). *P<0.05, **P<0.05 vs vehicle control (ANOVA followed by Dunnets Post Hoc test).

FIG. 3B shows the effect of PD98059-nanoparticle on induction of apoptosis of breast cancer (MDA-MB231) and melanoma (B16/F10) cell lines after 48 h of incubation. Data represents mean±SEM (n=3). *P<0.05, **P<0.05 vs vehicle control (ANOVA followed by Dunnets Post Hoc test).

FIG. 4 shows the mechanisms underlying the effect of PD98059-nanoparticle in vitro. Expression and phosphorylation status of ERK1/2 in B16/F10 and MDA-MB231 cells. Western blots and graph quantifying levels of p-ERK1/2 vs. total ERK in cells treated with PD-NPs (of two distinct size ranges >100 nm (big PD-nano) or <100 nm (small PD-nano)).

FIGS. 5A and 5B shows the effect of combination therapy of PD98059-NP with cisplatin inhibits B16/F10 melanoma in xenograft mouse model. FIG. 5A shows the tumor volume of B16/F10 melanoma in different treatment groups comparing the effects of PD98059-NP+ cisplatin, PD98059-NP, free PD98059, cisplatin. Control group received saline. FIG. 5B shows the body weight in different treatments as a measure of gross toxicity. For combination therapy, PD98059 was administrated (intravenous) on days 5, 8, and 11 (black arrows); cisplatin was administrated (intraperitoneal) on days 6, 9, and 12 (red arrows). Results are means±s. e.m. #P<0.05 vs free PD98059, *P<0.05 vs cisplatin alone (ANOVA followed by Newman Keuls Post Hoc test).

FIG. 6A is a schematic representation of the synthesis of LY294002 encapsulated nanoparticles by emulsion-evaporation technique. Typically poly (lactic-co-glycolic) acid (PLGA) having molecular weight 66 kD and LY294002 were dissolved in acetone:methanol (5:1, v/v) and added into 2% aqueous PVA solution to form a mini-emulsion. This mini-emulsion was added into 0.2% aqueous PVA solution. The solvent was evaporated and LY294002 encapsulated nanoparticles were isolated by ultracentrifugation at 80,000×g.

FIG. 6B shows results of TEM analysis of nanoparticles. The nanoparticles were fixed in gluteraldehyde, paraformaldehyde and sucrose in sodium cacodylate buffer, stained with 0.5% uranyl acetate and embedded in epon-812 resin. Sections were cut on a Leica ultra cut UCT at a thickness of 70 nm using a diamond knife. From the TEM image, the size range of spherical nanoparticles was found to be 60-120 nm in diameter.

FIG. 6C shows release kinetics of LY294002 from the nanoparticles. Lyophilized LY294002 encapsulated nanoparticles were suspended in PBS buffer and sealed in 1000 Da MWCO dialysis bag. Released LY294002 was quantified by reverse phase HPLC using dC18 column (4.6×150 mm) using acetonitrile:water (80:20) as mobile phase at retention time t=4.8 min at characteristics wave length e=298 nm. The values on the Y-axis represent the area under the curve, which is directly proportional to the concentration of released LY294002.

FIG. 7. Effect of NP-LY on viability of cancer cells. Breast adenocarcinoma (MDA-231), Lewis lung carcinoma (LLC) and melanoma (B16-F10) cells were plated on 96-well plates in the presence or absence of either free drug (LY) or LY-encapsulated nanoparticles (NP-LY). Cells were subjected to incubation with the drugs in a time- and concentration-dependent manner. At 24, 48 and 72 hours, the proportion of live cells remaining were quantified using the MTS assay. Data represents mean±SEM from atleast independent triplicates. ^{#}P<0.05 compared with vehicle-treated control cells.

FIG. 8A shows downstream activity in cancer cells. MDA-MB-231 and B16-F10 cells treated with LY or NP-LY for 24 hrs were subjected to immunoblotting against the phosphorylated (Phospho.) or total form of AKT.

FIG. 8B shows FACS analysis of cells treated with LY or NP-LY. MDA-MB-231 and B16/F10 cells were treated with LY or NP-LY for 48 hrs and then subjected to FACS analysis. Percentages of early and late apoptosis stages were quantified using the Annexin V-FITC/propidium iodide FACS assay. Cells were gated into four quadrants based on red (FL2-H) versus green (FL1-H) fluorescence, and the percentage of cells in each quadrant, representing a different apoptotic stage, is shown. Data shown are representatives from independent triplicates.

FIGS. 9A-9C shows the effect of NP-LY on angiogenesis in vitro. FIG. 9A shows Western analysis of HUVEC treated with LY or NP-LY for 24 hrs, followed by 15 min of VEGF. Representative and mean values of phosphorylated and total AKT optical densities are shown in the bar graph. FIG. 9B shows results of the MTS assays. HUVEC in 96-well plates were pretreated with various doses of free LY or NPLY for 1 hr, followed by the addition of FGF for up to 48 hrs, after which time the proportion of live cells remaining were quantified using the MTS assay. FIG. 9C shows the effects of LY and NP-LY on HUVEC tube formation. Effects were quantified by seeding cells on matrigel in the presence or absence of the drugs for 24 and 48 hrs. Mean values were quantified using three morphometric analyses. Data represents mean±SEM from atleast independent triplicates. For FIGS. 9A-9C, ^{#}P<0.05 compared with untreated control cells.

### DETAILED DESCRIPTION OF THE INVENTION

Cancer is the second leading cause of mortality in the United States, with an estimated 1,444,180 new cases and 565,650 deaths in 2008 [1]. Cytotoxic agents, which are used in standard chemotherapy, non-specifically target all dividing cells resulting in dose-limiting toxicities. There is an urgent need to develop novel strategies that are more specifically targeted against the tumor.

The mitogen activated protein kinase (MAPK) pathway comprising of RAS, RAF, MEK and ERK has been implicated in most human tumors, often through gain of function mutations in RAS and RAF family [2-3]. Indeed, RAS mutations are found in 30% of all cancer, and are in particular common in pancreatic cancer (90%) [4], colon cancer (50%) [5], while RAF mutations are prevalent in melanomas (63%) [6] and ovarian cancer (36%) [7]. As a result the MAPK pathway has evolved as a focus of intense investigation for developing small molecule inhibitors as targeted therapeutics. Many of these small molecule inhibitors are currently in clinical trials and have shown target suppression and tumor inhibition in Phase I studies (4).

Another emerging strategy for targeted chemotherapy is to harness nanovectors for preferential delivery of drugs into the tumor (8). A wide range of nanovectors, including liposomes, micelles, polymeric nanoparticles, silicon and gold nanoshells, polymeric dendrimers, and carbon-based nanostructures, have been used for drug delivery to the tumor [9]. Functionalizing the nanoparticles with polyethylene glycol prevents adsorption of proteins and biofouling and subsequent opsonization by the reticuloendothelial system, thereby conferring long-circulating property to the nanoparticles [10]. Furthermore, it is well established that long-circulating nanoparticles preferentially localize to the tumors [11] as a result of the enhanced permeation and retention (EPR) effect arising from unique ‘leaky’ vasculature of the tumor and the impaired lymphatic drainage [12]. Indeed, a nanoliposomal formulation of cisplatin was shown to attain 10-200 fold increased drug concentration in the tumors during a Phase-I clinical trial [13]. As compared with standard liposomal or protein carrier-based nanoplatforms that have limited control over drug release, controlled release drug delivery systems have the potential to induce standardized and durable clinical responses. Controlled release polymeric drug delivery based on polymer-drug conjugates are currently in clinical trials and target the tumors by passive delivery through the EPR effect [9].

Interestingly, while extensive studies have been done on delivering cytotoxic agents to solid tumors using nanovectors, to the best of our knowledge, no studies have yet been done on combining targeted therapeutics with nanoparticle-based tumor targeting. In this study, we integrated a PLGA-based nanoparticle with an extensively characterized selective inhibitor, PD98059 [14], to perturb the MAPK signaling pathway. A limitation of PLGA as a carrier is the linearity of the polymer, which results in low loading efficiency. To overcome this limitation we engineered a hexadentate-variant of PLGA, and engineered 80-140 nm nanoparticles with a 20 fold increase in drug loading. We observed that the nanoparticle formulation enables sustained drug release, which results in inhibition of phosphorylation of ERK, a downstream signal in the MAPK signal transduction cascade. This translates into inhibition of tumor cell proliferation and induction of apoptosis. Furthermore, we demonstrate that a nanoparticle-enabled targeting of the MAPK pathway in vivo enhances the antitumor effect as compared with free PD98059, and dramatically synergizes with cisplatin, a first line therapy for most cancers. Our results open up the exciting possibility of harnessing nanovectors for modulating oncogenic pathways using targeted therapeutics.

The invention is directed to modified polymers with increased drug-loading including compounds of formula (I):

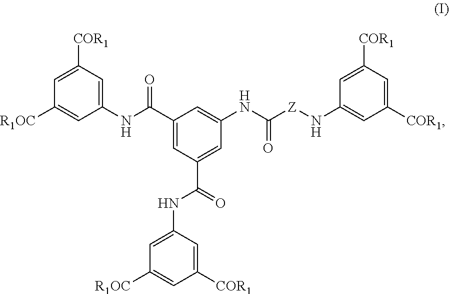

wherein Z is a polymer having molecular weight from 1-15 kDa;

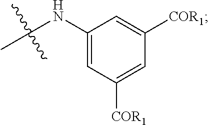

R₁ are independently H, R₂, OH, O-alkyl, —O—R₂, NH—R₂, -linker-R₂, or and
R₂ are independently one or more therapeutic agents.

The term “polymer”, as used herein, refers to a polymeric compound prepared by polymerizing monomers, whether of the same or a different type. The term “polymer” thus comprises, homopolymers, copolymers, block copolymers. The term “homopolymer” refers to polymers prepared from only one type of monomer. The term “copolymer”, as used herein, refers to polymers prepared by the polymerization of at least two different types of monomers. Preferably the polymer is a biocompatible and/or biodegradable polymer. The term “biocompatible” is used herein to refer to polymers that interacts with the body without undesirable aftereffects. The term “biodegradable” is used herein to mean capable of being broken down into innocuous products in the normal functioning of the body.

Suitable polymers include, by way of example, cellulose acetates (including cellulose diacetate), ethylene vinyl alcohol copolymers, hydrogels (e.g., acrylics), polyacrylonitrile and the like. Preferably, the biocompatible polymer is also noninflammatory when employed in situ.

One preferred polymer is poly(lactic-co-glycolic acid) (PLGA). The PLGA

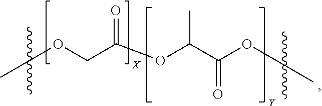

can be represented by the formula (II): wherein the ratio of monomers X and Y ranges from 1:10 to 10:1. In certain embodiments, the ratio of monomers X and Y is from 25:75 to 75:25. In a preferred embodiment, the ratio of monomers X and Y is 50:50.

In some embodiments, Z is a polymer having a molecular weight from 3-8 kDa. In a preferred embodiment, Z is polymer having a molecular weight of 4 kDa. In another preferred embodiment, Z is polymer having a molecular weight of 7 kDa.

Generally, at least one (e.g., 1, 2, 3, 4, 5, 6, 7, 8, 9, 10 or more) R₂ are present in a modified polymer of formula (I).

Linkers may be polymers, amino acid residues, alkyl groups or the like known in the art. The linkers may be cleavable depending on the desired use. Non-limiting examples are found in patent publication WO/2008/083312 and references therein.

In certain embodiments, R₂ is a therapeutic agent with an amine group. In some other embodiments, R₂ is a therapeutic group with a carboxyl and/or hydroxyl group.

As used herein, the term “therapeutic agent” refers to a substance used in the diagnosis, treatment, or prevention of a disease. Any therapeutic agent known to those of ordinary skill in the art to be of benefit in the diagnosis, treatment or prevention of a disease is contemplated as a therapeutic agent in the context of the present invention. Therapeutic agents include pharmaceutically active compounds, hormones, growth factors, enzymes, DNA, plasmid DNA, RNA, siRNA, viruses, proteins, lipids, pro-inflammatory molecules, antibodies, antibiotics, anti-inflammatory agents, anti-sense nucleotides and transforming nucleic acids or combinations thereof. Any of the therapeutic agents may be combined to the extent such combination is biologically compatible.

Exemplary therapeutic agents include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

Therapeutic agents also include chemotherapeutics known in the art, non-limiting examples include Actinomycin D, Adriamycin, Alkeran, Ara-C, Avastin, BiCNU, Busulfan, Carboplatinum, CCNU, Cisplatinum, Cytoxan, Daunorubicin, DTIC, 5-FU, Fludarabine, Gemcitabine, Herceptin, Hydrea, Idarubicin, Ifosfamide, Irinotecan, Leustatin, 6-MP, Methotrexate, Mithramycin, Mitomycin, Mitoxantrone, Navelbine Nitrogen Mustard Rituxan, 6-TG, Taxol, Taxotere, Topotecan, Velban, Vincristine, and VP-16.

In certain embodiments, the therapeutic agent is a kinase inhibitor. In a preferred embodiment, the kinase inhibitor is PD98059. In certain embodiments, kinase inhibitor blocks one or more of VEGFR, PI3K, MET, EGFR, PDGFR, or erb2.

In certain embodiments, the therapeutic agent is Lapatinib, Erlotinib, Vatalanib, Gefitinib, Nilotinib, Sunitinib, or TNP-470.

Non-limiting examples of therapeutic agents include anti-thrombogenic agents; antioxidants; angiogenic and anti-angiogenic agents and factors; anti-proliferative agents (e.g., agents capable of blocking smooth muscle cell proliferation); anti-inflammatory agents; calcium entry blockers; antineoplastic/antiproliferative/anti-mitotic agents (e.g., paclitaxel, doxorubicin, cisplatin); antimicrobials; anesthetic agents; anti-coagulants; vascular cell growth promoters; vascular cell growth inhibitors; cholesterol-lowering agents; vasodilating agents; agents which interfere with endogenous vasoactive mechanisms; and survival genes which protect against cell death. Therapeutic agents are described in co-pending U.S. patent application Ser. No. 10/615,276, filed on Jul. 8, 2003, and entitled “Agent Delivery Particle”, which is incorporated herein by reference.

Another aspect of the invention is directed to nanoparticle drug delivery systems including a PLGA-b-PEG block copolymer; and a stabilizer. Stabilizers include polyvinyl alcohol (PVA) and polyvinyl pyrrolidone (PVP) and other well known in the art.

In certain embodiments, the modified polymer is substantially the same as described herein.

In certain embodiments, the nanoparticle drug delivery system described herein includes one or more additional therapeutic agents.

In certain embodiments, the additional therapeutic agent is at least one chemotherapeutic agent covalently bound to the PLGA.

In certain embodiments, the additional therapeutic agent is doxorubicin, a taxane, a podophyllotoxin, vinca alkaloids, or methotrexate.

In a preferred embodiment, the additional therapeutic agent is a PLGA-LY294002-PVA nanoparticle wherein the LY294002 is not covalently bound to the PLGA.

In a preferred embodiment, the stabilizer is polyvinyl alcohol (PVA).

Yet another aspect of the invention is directed to drug delivery systems including PLGA-b-PEG block copolymer polyvinyl alcohol (PVA) nanoparticle; and the modified polymer substantially as described herein.

The present invention may be defined in any of the following numbered paragraphs:

1. A modified polymer with increased drug-loading comprising:
a compound of formula (I):

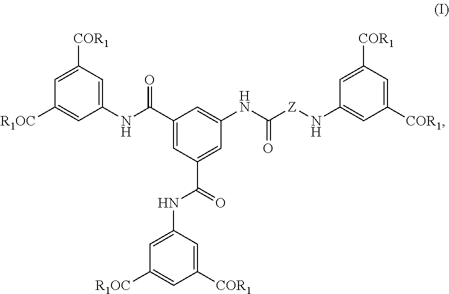

wherein Z is a poly(lactic-co-glycolic acid) (PLGA) polymer having molecular weight from 1-15 kDa;
   R₁ are independently H, R₂, OH, O-alkyl, —O—R₂, NH—R₂, -linker-R₂, or

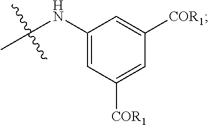

and
R₂ are independently one or more therapeutic agents.

2. The modified polymer of paragraph 1, wherein the PLGA polymer has a molecular weight from 3-8 kDa.

3. The modified polymer of any of paragraphs 1-2, wherein the PLGA polymer has a molecular weight of 4 kDa.

4. The modified polymer of any of paragraphs 1-3, wherein the PLGA polymer has a molecular weight of 7 kDa.

5. The modified polymer of any of paragraphs 1-4, wherein PLGA polymer is represented by the formula (II):

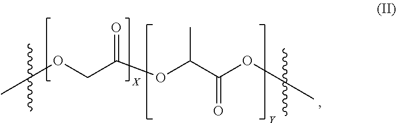

wherein the ratio of monomers X and Y ranges from 1:10 to 10:1.

6. The modified polymer of paragraph 5, wherein the ratio of monomers X and Y is from 25:75 to 75:25

7. The modified polymer of paragraph 5 or 6, wherein the ratio of monomers X and Y is 50:50.

8. The modified polymer of any of paragraphs 1-7, wherein R₂ is a therapeutic agent with an amine group.

9. The modified polymer of any of paragraphs 1-8, wherein said therapeutic agent is a kinase inhibitor.

10. The modified polymer of paragraph 9, wherein said kinase inhibitor is PD98059.

11. The modified polymer of paragraph 9, wherein said kinase inhibitor blocks one or more of VEGFR, PI3K, MET, EGFR, PDGFR, or erb2.

12. The modified polymer of any paragraph 1-8, wherein said therapeutic agent is Lapatinib, Erlotinib, Vatalanib, Gefitinib, Nilotinib, Sunitinib, or TNP-470.

13. A nanoparticle drug delivery system comprising:
a PLGA-b-PEG block copolymer; and
a stabilizer.

14. The nanoparticle drug delivery system of paragraph 13 further comprising the modified polymer of paragraphs 1-12.

15. The nanoparticle drug delivery system of paragraph 13 or 14 further comprising one or more additional therapeutic agents.

16. The nanoparticle drug delivery system of paragraph 15, wherein the additional therapeutic agent is at least one chemotherapeutic agent covalently bound to the PLGA.

17. The nanoparticle drug delivery system of paragraph 16, wherein the additional therapeutic agent is doxorubicin, a taxane, a podophyllotoxin, vinca alkaloids, or methotrexate.

18. The nanoparticle drug delivery system of paragraph 15, wherein the additional therapeutic agent is a PLGA-LY294002-PVA nanoparticle wherein the LY294002 is not covalently bound to the PLGA.

19. The nanoparticle drug delivery system of paragraph 13, wherein the stabilizer is polyvinyl alcohol (PVA).

20. A drug delivery system comprising:

PLGA-b-PEG block copolymer polyvinyl alcohol (PVA) nanoparticle; and the modified polymer of paragraphs 1-12.

The following examples demonstrate the preparation of compounds according to this invention. The examples are illustrative, and are not intended to limit, in any manner, the claimed invention.

### EXAMPLES

### Example 1

### PD98059-PLGA Conjugates

### Materials and Reagents

All the reagents were purchased from Aldrich, Fluka, Fisher, Tocris, Nanocs unless otherwise stated and used without any further purification. All the solvents used for synthesis were dry solvents and all the synthetic reactions were carried out under nitrogen atmosphere unless otherwise stated. All the dry solvents were purchased from Aldrich and used without any further distillation. The poly (lactic-co-glycolic acid) (Mw˜4 kDa) having a lactic/glycolic molar ratio of 50/50 is a generous gift from the Tempo Pharmaceutical. The ¹H and ¹³C NMR spectra were recorded using Varian Mercury 300 MHz machine at room temperature. UV-VIS spectra were measured using Shimadzu UV-2450 UV-VIS Spectrophotometer. Malvern Nanozetasizer was used to measure Dynamic Light scattering. TEM was measured by Jeol E M. CellTiter 96 AQueous One Solution Cell Proliferation (MTS) Assay was obtained from Promega Corporation (Madison, Wis.). AnnexinV-Alexa Fluor 488 and LysoTracker Red probe were from Invitrogen (Carlsbad, Calif.). Polyclonal antibodies specific for actin, as well as for the phosphorylated form of ERK1/2 (pi-ERK1/2) was purchased from Cell Signaling Technology (Danvers, Mass.), whereas anti-ERK1/2 antibodies were obtained from Santa Cruz Biotechnology (Santa Cruz, Calif.).

General Procedure for Conjugating PD98059 with Modified PLGA:

Modified hexadentate PLGA was synthesized as described below. PLGA (1 equiv) was dissolved in dimethylformamide (DMF) under nitrogen atmosphere in a round bottom flask. Into it HBTU (1.5 equiv per carboxylic acid group on polymer) was added followed by DIPEA (2 equiv per carboxylic acid group on polymer). The reaction mixture was stirred at room temperature for 15 minutes. The pale brown color indicates the activation of the carboxylic acid of PLGA. Activated PLGA was then added into PD98059 (1 equiv per carboxylic acid group on polymer) solution in dry DMF and the reaction mixture was stirred at room temperature for 24 h. The PLGA-PD98059 conjugate was precipitated from the reaction mixture by diethyl ether (30 mL) and centrifuged at 3220×g for 30 minutes. The precipitated polymer was collected and washed repeatedly with diethyl ether to remove the excess reagents. Finally the polymer was dried under vacuum for 24 h to obtain the conjugated product. Characterization of PLGA-6(PD98059) conjugate (9): UV-VIS Spectrum: UV-VIS spectrum of the product shows two peaks at wavelength λ=350 nm and 297 nm which are the characteristics peaks for PD98059 molecule. ¹H NMR (300 MHz): δ (ppm)=8.8-8.5 (m, aromatic proton), 8.2-8.1 (m, aromatic proton), 7.7-7.6 (m, aromatic proton), 7.5-7.4 (m, aromatic proton), 6.7-6.6 (m, olefin proton of PD98059), 5.2-5.1 (m, polymer protons), 4.9-4.6 (m, polymer protons), 3.7-3.6 (m, —OCH₃ protons of PD98059), 1.61-1.56 (m, polymer proton).

### Synthesis of PEGylated Nanoparticles:

A mixture of 20 mg PLGA-PD98059 and 4 mg PLGA-PEG conjugates were dissolved completely in 1.25 mL acetone and 0.25 mL methanol. The entire solution was emulsified into 12.5 mL 2% aqueous solution of PVA (80% hydrolyzed, Mw˜9000-10,000) by slow injection with constant homogenization using a tissue homogenizer. This mini emulsion was added to a 50 mL 0.2% aqueous solution of PVA (80% hydrolyzed, Mw 9000-10,000) with rapid mixing for 4 h at room temperature to evaporate any residual acetone or methanol. Nanoparticles were recovered by ultracentrifugation at 80,000^{x}g. Sizing and morphological analysis was performed by dynamic light scattering (Malvern Nanozetasizer) and transmission electron microscopy (TEM). The nanoparticles were washed thoroughly with double distilled water to remove excess PVA before preparing the sample for TEM. For TEM, the nanoparticles were fixed in 2.5% gluteraldehyde, 3% paraformaldehyde with 5% sucrose in 0.1M sodium cacodylate buffer (pH=7.4), embedded in low temperature agarose and post fixed in 1% OsO₄ in veronal-acetate buffer. The sample was stained in block over night with 0.5% uranyl acetate in veronal-acetate buffer (pH=6.0); then dehydrated and embedded in epon-812 resin. Sections were cut on a Leica ultra cut UCT at a thickness of 70 nm using a diamond knife, stained with 2.0% uranyl acetate followed by 0.1% lead citrate and examined using a Philips EM410.

### Synthesis of Gold NP Decorated PEGylated NP:

50 mg of lyophilized biotinylated NP (11) was suspended in 500 μL PBS and 1 mL of streptavidine coated gold NP (0.01% gold) was added and incubated at 30° C. for 48 h by gentle shaking. The gold NP coated PLGA-PEG-Biotin NP was isolated by ultra centrifugation at 80,000×g speed. The excess streptavidine-gold NPs were removed by through washing using double distilled water. The gold NP coated pegylated NPs were suspended in 100 μL double distilled water and TEM was measured.

### Physicochemical Release Kinetics Characterization:

PD98059 loaded nanoparticles were suspended in 1 mL of hypoxic-cell lysate (from MDA-MB-231, LLC and B16-F10 cell lines) and sealed in a dialysis bag (MWCO 1000 Da). The dialysis bag was incubated in 1 mL of PBS buffer at room temperature with gentle shaking. 10 μL of aliquot was extracted from the incubation medium at predetermined time intervals, dissolved in 90 μL DMF and released PD98059 was quantified by UV-VIS spectroscopy at characteristic wavelength of PD98059, λ=297 nm. After withdrawing each aliquot the incubation medium was replenished by 10 μL of fresh PBS.

### Cell Culture:

Cancer cells were obtained from American Type Tissue Culture Collection (Rockville, Md.) and were maintained in DMEM supplemented with 10% FBS and antibiotic/antimycotic (all from Invitrogen). MDA-MB-231 is a human breast human adenocarcinoma cell line whereas B16-F10 and LLC are derived from mouse melanoma and Lewis lung carcinomas models, respectively. All cells were grown on 100 mm dishes and subcultured using trypsin (0.25%) and EDTA (0.01%) treatment and replated at different ratios depending on the experiment. Cells were switched serum reduced to 1% prior to drug addition, in order to quantitate the effect of the drug proper. The drugs used throughout experiments consisted of the free drug, PD98059 (PD) or PD98059-conjugated nanoparticles (NP) of two different sizes, namely over or under 100 nm (NP>100 nm or NP<100 nm, respectively). DMSO was used as solvent.

### Cell Viability Assay:

Cancer cells in 96-well plates were incubated with various doses PD or NP for 24, 48 and 72 hrs. The percentage of viable cells was then quantified with 3-(4,5-Dimethylthiazol-2-yl)-5-(3-carboxymethoxyphenyl)-2-(4-sulfophenyl)-2H-tetrazolium (MTS) from the CellTiter 96 AQueous One Solution kit. MTS is reduced by mitochondrial dehydrogenases of live cells, yielding a colored adduct that can be read spectrophotometrically. Briefly, the cells were washed with PBS, incubated with 0.3 mg/ml of MTS, in basal medium without phenol red, for 4 hrs at 37° C. and absorbance was then measured at 490 nm in a plate reader (Versamax, Molecular Devices, Sunnyvale, Calif.). Final absorbance, corresponding to cell proliferation, was plotted after removing background values from each data point.

### Apoptosis Study:

Cells grown in 6-well plates were treated with drugs for 48 h, and incubated with 5 μL AnnexinV-Alexa Fluor 488 in binding buffer (10 mM HEPES, 140 mM NaCl, 2.5 mM CaCl₂, pH 7.4) for 15 min in the dark, according to the manufacturer's protocol. Cells were then washed with binding buffer, counterstained with propidium iodide and immediately processed for FITC and propidium iodide staining using a Becton Dickinson FACSCalibur flow cytometer (excitation 488 and 585 nm, respectively). AnnexinV-Alexa Fluor 488, propidium iodide or both were omitted for the negative controls.

### Drug Uptake and Metabolism:

LysoTracker probes are weakly basic amines, which accumulate in the acidic compartments of live cells and can hence be used to track drug uptake and metabolism. MDA-MB-231 and B16-F10 cells were seeded on glass coverslips in 24-well plates until subconfluency, and then treated with 5.6 mg/ml FITC-conjugated nanoparticles (FITC-NP) for a time-course ranging from 30 min to 24 hrs. At the indicated times, cells were washed twice in PBS and incubated in LysoTracker Red (Ex: 577 nm; Em: 590 nm) for 30 min at 37° C. Cells were then washed again, fixed in 4% paraformaldehyde and mounted using Prolong Gold antifade reagent (Invitrogen). Images taken in 3 random fields were captured at 20× and 40× using an inverted microscope (Nikon Eclipse, Melville, N.Y.) equipped with blue and green filters in order to visualize FITC-NP and LysoTracker Red fluorescence, respectively. Cells incubated either only FITC-NP or Lysotracker red served as negative controls.

### Immunoblotting of Cell Extracts.

Cells were washed twice with PBS and directly lysed in 3× loading buffer containing 12% sodium dodecyl sulfate, 15% 2-mercaptoethanol, 1 mM sodium orthovandate and protease inhibitor cocktail tablets from Roche Applied Science (Indianapolis, Ind.). Cells were further homogenized by passing the lysates 3 times through an insulin needle. Samples were then heated for 5 min at 100° C. and equal amounts loaded onto tris-glycine SDS-polyacrylamide gels. Proteins were electrophoretically transferred onto polyvinylidene difluoride membranes, blocked for 1 h with 7% non-fat dry milk, and subsequently incubated overnight at 4° C. with primary antibodies directed against the phosphorylated or total forms of ERK1//2 and AKT. Proteins were detected with horseradish peroxidase-conjugated anti-rabbit secondary antibodies and Lumi-LightPLUS Western Blotting Substrate (Roche Applied Science). The blots were developed using GeneSnap and optical densities off the protein bands quantified using GeneTools (both from SynGene, Frederick, Md.). Predetermined molecular weight standards were used as markers. Proteins were normalized against actin.

### In Vivo Murine B16/F10 Melanoma Tumor Model.

Male C57/BL6 mice (20 g) were injected with 5×10⁵ BL6/F10 melanoma cells into the flanks. The drug therapy was started after the tumors attained volume of 25 mm3. The animals were intravenously injected with free PD98059 or PD98059-nanoparticles such that the total dose of PD98059 was 5 mg/kg of PD98059 (administered by tail vein injection). A batch of PD98059 (free or as nanoparticle)-treated animals were subsequently injected with cisplatin (2.5 mg/kg), which was administered intraperitoneally after 12 hours following the PD98059 dosing. The total volume of injection was 100 μl. The tumor volumes and body weights were monitored on a daily basis. The animals were sacrificed at predefined time points. The organs (liver, lung, spleen, kidney and tumor) were harvested immediately following sacrifice and divided into equal parts and stored at −80° C. for further analysis.

### Statistical Analysis:

All results were expressed as mean±SEM of at least triplicate samples. Statistical comparisons were obtained using one-way analysis of variance. Probability (p) values less than 0.05 were considered significant

### Synthesis of PLGA-PD98059 Conjugate (2):

PLGA (50 mg, 0.012 mmol) was dissolved in 1 mL dimethylformamide (DMF) under nitrogen atmosphere in a round bottom flask. Into it HBTU (7 mg, 0.0178 mmol) was added followed by DIPEA (5 uL, 0.0238 mmol). The reaction mixture was stirred at room temperature for 15 minutes. The pale brown color indicates the activation of the carboxylic acid of PLGA. Activated PLGA was then added into PD98059 (10 mg, 0.036 mmol) solution in 1 mL dry DMF and the reaction mixture was stirred at room temperature for 24 h. The PLGA-PD98059 conjugate was precipitated from the reaction mixture by diethyl ether (30 mL) and centrifuged at 3220×g for 30 minutes. The precipitated polymer was collected and washed repeatedly with diethyl ether to remove the excess reagents. Finally the polymer was dried under vacuum for 24 h to obtain the conjugated product.

UV-VIS spectrum of the product shows two peaks at wavelength λ=350 nm and 297 nm which are the characteristics peaks for PD98059 molecule. ¹H NMR (300 MHz): δ (ppm)=8.0-7.9 (m, aromatic protons), 7.5-7.4 (m, aromatic protons), 5.25-5.21 (m, polymer protons), 4.91-4.68 (m, polymer protons), 2.98 (s, —OCH₃, proton), 1.61-1.56 (m, polymer proton). ¹³C NMR (75 MHz): δ (ppm)=181.1, 179.5, 169.5, 169.4, 166.6, 154.8, 152.3, 148.3, 144.4, 143.4, 132.3, 127.9, 116.7, 113.1, 111.8, 110.2, 69.3, 69.1, 60.9, 16.8.

### Synthesis of Activated PLGA (4):

PLGA (50 mg, 0.012 mmol) was dissolved in 1 mL of dichloromethane (DCM) and cooled to 0° C. Into the reaction mixture p-nitrophenylchloroformate (7 mg, 0.033 mmol) and pyridine (5 μL, 0.056 mmol) were added and the reaction was stirred at room temperature for 4 h. The reaction was diluted with DCM and quenched with 0.1 N HCl solution. The organic layer was extracted by DCM (2×20 mL), washed with brine and dried over anhydrous Na₂SO₄. The solvent was evaporated to obtain the activated polymer 4. UV-VIS Spectrum: UV-VIS spectrum of the product shows a peak at λ=267 nm which is the characteristics peak of p-nitrophenyl moiety. ¹H NMR (300 MHz): δ (ppm)=8.32-8.22 (m, aromatic proton), 8.11-8.04 (m, aromatic proton), 7.49-7.44 (m, aromatic proton), 7.32-7.19 (m, aromatic proton), 5.25-5.21 (m, polymer protons), 4.91-4.68 (m, polymer protons), 1.61-1.56 (m, polymer proton). ¹³C NMR (75 MHz): δ (ppm)=169.8, 166.8, 126.6, 125.8, 122.7, 116.0, 110.4, 69.7, 61.4, 54.4, 30.1, 17.1.

### Synthesis of Polymer 6:

Polymer 4 (100 mg, 0.024 mmol) and 5-aminoisophthalic acid were dissolved in 1 mL dimethylformamide (DMF). Into it diisopropylethyl amine (DIPEA) (17 μL, 0.095 mmol) was added and the reaction mixture was stirred at room temperature for 24 h. As soon as DIPEA added the reaction mixture turned to yellow, which indicates the liberation of p-nitrophenoxide anion in the solution. The polymer was precipitated out from the reaction mixture by adding diethyl ether (40 mL), centrifuged at 3220×g for 40 minutes, washed thoroughly with diethyl ether (5×5 mL) to remove excess reagents. The product polymer was dried under vacuum to obtain product 6. UV-VIS Spectrum: UV-VIS spectrum of the product shows a peak at λ=250 nm which is the characteristics peak of 5-aminoisophthalic acid moiety. ¹H NMR (300 MHz): δ (ppm)=8.82-8.80 (m, aromatic proton), 8.75-8.70 (m, aromatic proton), 7.62-7.58 (m, aromatic proton), 5.25-5.21 (m, polymer protons), 4.91-4.68 (m, polymer protons), 1.61-1.56 (m, polymer proton). ¹³C NMR (75 MHz): δ (ppm)=170.4, 169.2, 154.2, 126.0, 125.2, 122.9, 116.5, 110.4, 69.5, 61.0, 54.5, 16.0.

### Synthesis of Modified PLGA (8):

Polymer 6 (300 mg, 0.071 mmol) was dissolved in 2 mL DMF and the carboxylic acids were activated by N,N′-dicyclohexyl carbodiimide (DCC) (66 mg, 0.32 mmol) and N-hydroxy succinimide (NHS) (37 mg, 0.32 mmol) at room temperature for 18 h. The formation of insoluble dicyclohexyl urea (DCU) indicates the formation of the activated carboxylic acids. 5-aminoisophthalic acid (58 mg, 0.32 mmol) and DIPEA (74 uL, 0.43 mmol) were added and the reaction mixture was stirred at room temperature for another 24 h. DCU was filtered, washed thoroughly by DCM (5×5 mL). The solvent was evaporated and the polymer was precipitated out by diethyl ether (45 mL). The polymer was spun down at 3220×g for 40 minutes and dried under high vacuum to obtain polymer 8. UV-VIS Spectrum: UV-VIS spectrum of the product shows a peak at λ=250 nm which is the characteristics peak of 5-aminoisophthalic acid moiety. ¹H NMR (300 MHz): δ (ppm)=8.9-8.8 (m, aromatic protons), 8.7-8.6 (m, aromatic protons), 8.5-8.3 (m, aromatic protons), 5.25-5.21 (m, polymer protons), 4.91-4.68 (m, polymer protons), 1.61-1.56 (m, polymer proton).

### Synthesis of FITC-Labeled PLGA:

PLGA (50 mg) was dissolved in 750 μL dichloromethane. NHS (10 mg) and EDC (15 mg) were added into the reaction mixture and stirred at room temperature for 2 h. 6 mg FITC was dissolved in 25 μL dichloromethane and 25 μL pyridine. FITC solution in pyridine was added into the activated PLGA solution and the reaction mixture was stirred at 4° C. for 24 h in dark. The reaction was diluted with 50 mL DCM and quenched with 0.1 N HCl solution. The organic layer was extracted with DCM (20 mL×2), washed with water (10 mL×2), brine (20 mL) and dried over anhydrous sodium sulfate. The organic layer was filtered and evaporated to obtain the crude product. The PLGA-FITC conjugate was precipitated out from the crude product by addition of diethyl ether (40 mL). The polymer was centrifuged at 3220×g for 30 minutes. The supernatant was discarded and the polymer was washed thoroughly by diethyl ether (5 mL×3) and dried under vacuum overnight.

### Synthesis of PLGA-PEG Conjugate 10:

PLGA (50 mg, 0.012 mmol) was dissolved in DMF (1 mL). The carboxylic acid of PLGA was activated by HBTU (7.0 mg, 0.018 mmol) and DIPEA (9 μL, 0.05 mmol) for 10 minutes at room temperature. The pale brown color indicates the activation of the carboxylic acid of PLGA. The activated PLGA was then added into amino polyethylene glycol (PEG-NH₂) (36 mg, 0.018 mmol) solution in 1 mL dry DMF and the reaction mixture was stirred at room temperature for 24 h. The PLGA-PEG conjugate was precipitated out from the reaction mixture by adding diethyl ether (40 mL) and centrifuged at 3220×g for 30 minutes. The supernatant was discarded and the polymer was washed with diethyl ether (3×5 mL) to remove excess reagents. Finally the polymer was dried under vacuum for 24 h to obtain the conjugated product. The polymer was characterized by ¹H NMR spectroscopy. ¹H NMR (300 MHz): δ (ppm)=5.05-5.00 (m, PLGA-CH—), 4.78-4.66 (m, PLGA-CH₂—), 3.31-3.22 (m, PEG-CH₂—), 1.28 (s, PLGA-CH3-).

### Synthesis of PLGA-PEG-Biotin Conjugate 11:

PLGA (25 mg, 0.006 mmol) was dissolved in DMF (1 mL). The carboxylic acid of PLGA was activated by HBTU (4.0 mg, 0.009 mmol) and DIPEA (5 μL, 0.003 mmol) for 10 minutes at room temperature. The pale brown color indicates the activation of the carboxylic acid of PLGA. The activated PLGA was then added into amino biotin polyethylene glycol amine (Biotin-PEG-NH₂) (30 mg, 0.009 mmol) solution in 1 mL dry DMF and the reaction mixture was stirred at room temperature for 24 h. The PLGA-PEG-Biotin conjugate was precipitated out from the reaction mixture by adding diethyl ether (40 mL) and centrifuged at 3220×g for 30 minutes. The supernatant was discarded and the polymer was washed with diethyl ether (3×5 mL) to remove excess reagents. Finally the polymer was dried under vacuum for 24 h to obtain the conjugated product. The polymer was characterized by ¹H NMR spectroscopy. ¹H NMR (300 MHz): δ (ppm)=5.19-5.14 (m, PLGA-CH—), 4.88-4.75 (m, PLGA-CH2-), 4.70-4.69 (m, biotin protons), 3.68-3.61 (m, PEG-CH₂—), 3.48-3.41 (m, biotin protons), 3.12-3.11 (m, biotin protons), 1.57-1.52 (m, PLGA-CH₃—), 1.47-1.39 (m, biotin protons), 1.30-1.29 (m, biotin protons), 1.20-1.58 (m, biotin proton).

### Synthesis of the Nanoparticles:

Nanoparticles were formulated using an emulsion-solvent evaporation technique as described. 50 mg PLGA-PD98059 (or FITC-PLGA) conjugate was dissolved completely in 2.5 mL acetone and 0.5 mL methanol. The entire solution was emulsified into 25 mL 2% aqueous solution of PVA (80% hydrolyzed, Mw˜9000-10,000) by slow injection with constant homogenization using a tissue homogenizer. This mini emulsion was added to a 100 mL 0.2% aqueous solution of PVA (80% hydrolyzed, Mw˜9000-10,000) with rapid mixing for 4 h at room temperature to evaporate any residual acetone or methanol. Nanoparticle size fraction was recovered by ultracentrifugation at 20,000 and 80,000×g. Sizing was performed by dynamic light scattering (DLS) and transmission electron microscopy (TEM). The nanoparticles were washed thoroughly with double distilled water to remove excess PVA before preparing the sample for TEM.

### Synthesis and Characterization of PLGA-PD98059 Conjugates.

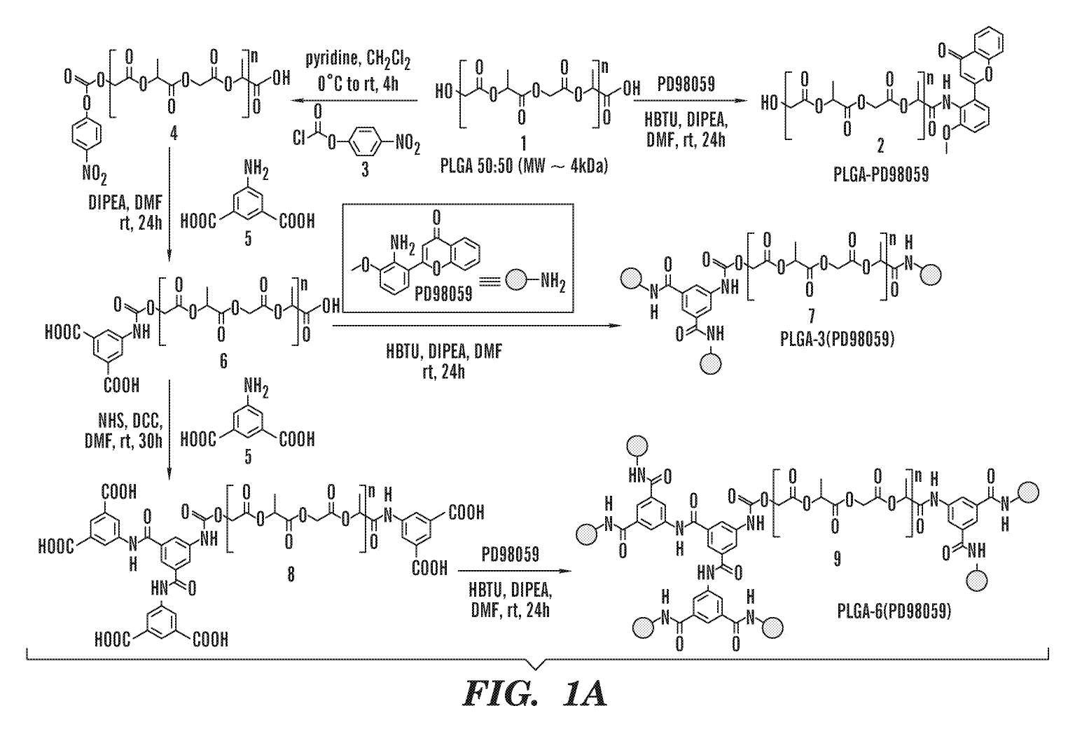

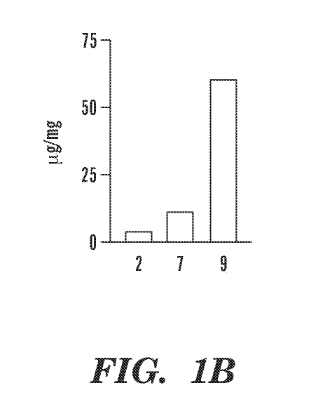

Nanoparticles engineered from biodegradable, biocompatible, and FDA-approved polymers offer the potential for rapid translation to the clinics. As a result, we decided to adapt poly(D,L-lactic-co-glycolic acid) (PLGA), a clinically approved material, as the base polymer to engineer the nanoparticles. As a proof of principle of nanoparticle-mediated mechanistic targeting, we selected PD98059 as the selective inhibitor to block MAPK signaling. In previous studies, PD98059 was shown to inhibit MEK with an IC50˜10 μM but had no inhibitory effects when tested against a panel of 18 other serine/threonine kinases [15]. To avoid the characteristics ‘burst’ release associated with nanoparticles and achieve a controlled release profile, PD98059 was conjugated to linear PLGA 5050 (1) using amide coupling reaction to obtain PLGA-PD98059 (1:1) conjugate (2) (FIG. 1A). The loading of PD98059 in this conjugate was determined to be 3.0 μg/mg (by UV-VIS spectroscopy at the characteristics wavelength of free PD98059 at λ=297 nm).

To optimize the loading, we modified the native PLGA (1) to a tricarboxylated PLGA (6) using a non-toxic 5-aminoisophthalic acid (5) by a two-step procedure. First, the terminal hydroxyl group of glycolic acid was activated using 4-nitrophenyl chloroformate (3) to obtain the activated PLGA (4), and then activated PLGA (4) was treated with 5-aminoisophthalic acid (5) in presence of diisopropylethyl amine (DIPEA) as base. The conjugation of PD98059 to the tri-carboxylic PLGA (6) gave PLGA-3(PD98059) (1:3) conjugate (7). Loading of PD98059 in conjugate 7 was determined to be 11.0 μg/mg. In order to further increase the loading capacity, we modified the tri-carboxylic PLGA (6) to hexa-carboxylic PLGA (8) by activating the carboxylic acids in polymer 6 using dicyclohexyl carbodiimide (DCC), N-hydroxy succinimide (NHS). The activated carboxylic acids were then reacted with 5-aminoisophthalic acid (5) in presence of DIPEA as base to obtain the hexa-carboxylic PLGA (8). The conjugation of PD98059 to hexa-carboxylic PLGA (8) afforded PLGA-6(PD98059) (1:6) conjugate (9). Loading of PD98059 in conjugate 9 was determined to be 60 μg/mg (FIG. 1B). The structure of molecules generated at each step was confirmed using spectroscopic and analytical methods [see supplementary information].

### Engineering PEG Functionalized PD98059-Loaded Nanoparticles.

Although the formation of nanoparticles from PLGA is well established, the inventors discovered that the aromatic modification of PLGA can lead to the formulation of spherical nanoparticles. Using a previously reported emulsion-solvent evaporation method led to formulation of nanoparticles (NPs) from the conjugate 9 (16). The surface morphology and size distribution of the nanoparticles were evaluated by transmission electron microscopy (TEM) (data not shown) and dynamic light scattering (DLS) experiments. From the TEM it was evident that the aromatic modification of native PLGA did not change the morphology of the NPs formed. From the DLS and TEM the size distribution of the NPs synthesized was found to be in the range 60-140 nm in diameter (data not shown).

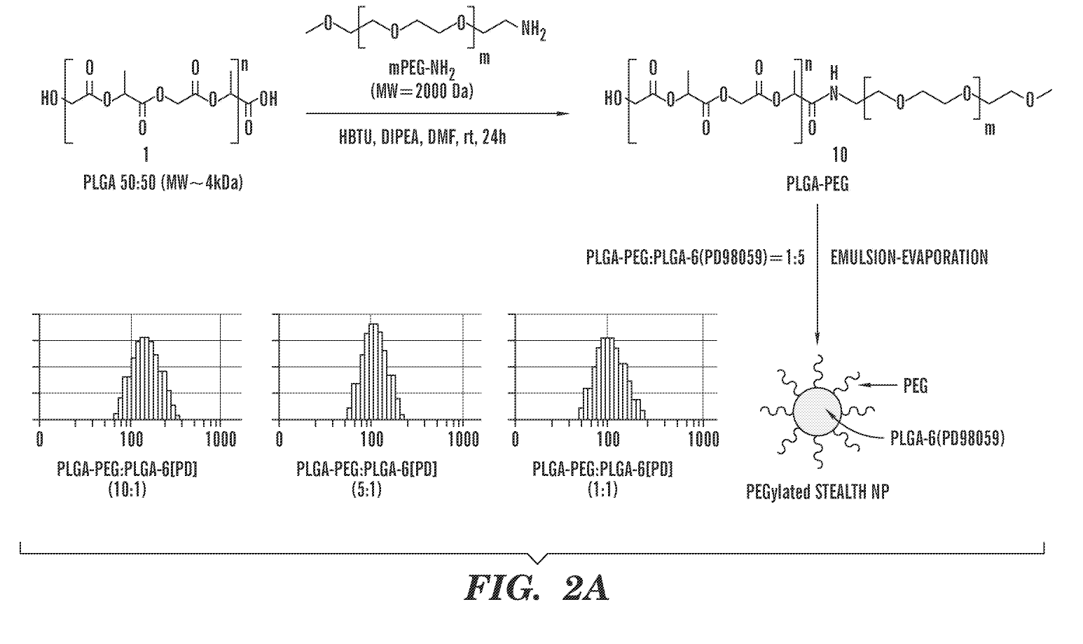

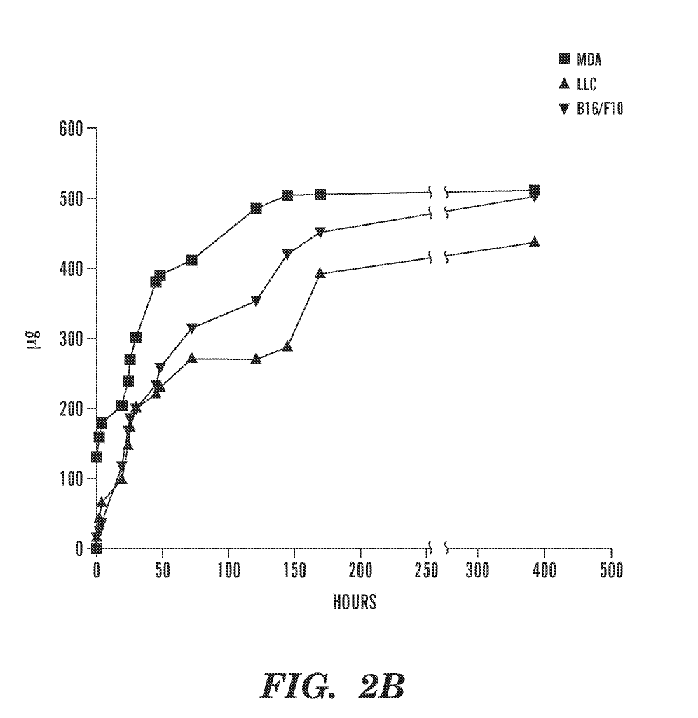

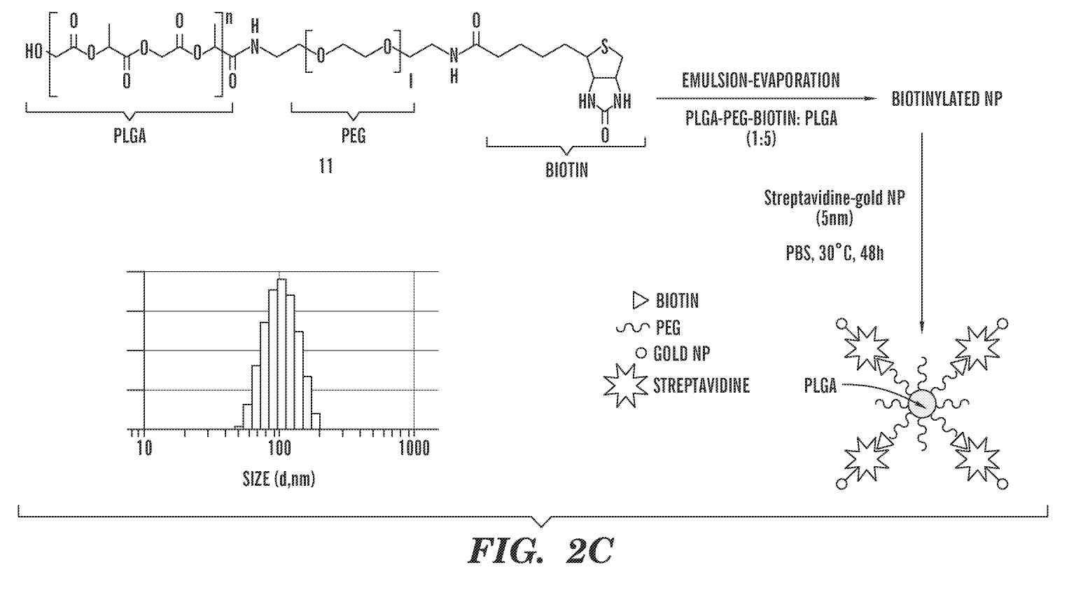

Nanoparticles whose surfaces were not modified to prevent absorption of opsonins are reportedly cleared rapidly by macrophages. It has been suggested that adsorption of plasma proteins depends primarily on the nanoparticle hydrophobicity and charge (10). Surface modification of the nanoparticle with polyethylene glycol (PEG) has been reported to decrease surface interactions with opsonins by steric repulsion [17]. Furthermore, PEG has exhibited excellent biocompatibility and is already approved by the FDA for human use [18]. To develop the ‘stealth’ nanoparticles to prevent their uptake by tissue macrophases and nontargeted cells, the inventors synthesized a PLGA-b-PEG block copolymer (10) by amide coupling of the carboxylic acid of PLGA with the amine group of 2 KDa amine ethylene glycol (m-PEG-NH₂) in presence of coupling reagent HBTU and DIPEA as base (FIG. 2A). The pegylated ‘stealth’ NPs were formulated using emulsion-solvent evaporation technique. To optimize the size and surface coverage of the NPs by polyethylene glycol (PEG) to provide a stealth capability, NPs using varying ratio of PLGA-b-PEG:hexadentate PLGA-PD98059 (PLGA-6(PD98059)) [1:10, 1:5 and 1:1 w/w] were formulated From DLS study, it was observed that the PLGA-b-PEG:PLGA-6(PD98059)=1:5 gave the most optimal size distribution (mean diameter=100-120 nm, FIG. 2A).

In order to test whether a ratio of 1:5 of PLGA-b-PEG:PLGA-6(PD98059) confers optimal surface coverage of the nanoparticles by PEG, a method based on the well validated streptavidin-biotin binding to visualize the PEG chains on the surface of the nanoparticles using TEM was developed. Biotin-labeled NPs from PLGA-b-PEG-Biotin conjugate (11) were first synthesized (FIG. 2C), and then nanoparticles using the PLGA-b-PEG-Biotin: PLGA in 1:5 ratio were formulated. The nanoparticles were then probed with streptavidin-gold nanoparticles (5 nm). The nanoparticle was then cross-sectioned, stained and visualized using TEM. The complexation of the gold-NPs at the periphery of the cross section of PLGA-PEG-NPs showed that most of the PLGA-NPs surface area was coated with biotinylated PEG. No such binding was observed with nanoparticles that were constructed with non-pegylated PLGA (data not shown).

In Vitro Release Kinetics of PD98059 from Nanoparticles.

Next controlled release kinetics of PD98059 from the nanoparticles were studied. To mimic the clinical situation, nanoparticles were incubated with tumor cell lysates, and the released PD98059 was dialyzed against 1 ml PBS. As shown in FIG. 2B, incubation with lysates of MDA-MB231, B16/F10 melanoma and Lewis Lung carcinoma (LLC) cells, resulted in a sustained release of PD98059 from the nanoparticles, with a faster release evident with the MDA-MB231 breast cancer cells followed by incubation with B16/F10 and LLC.

### In Vitro Cellular Cytotoxicity Assay:

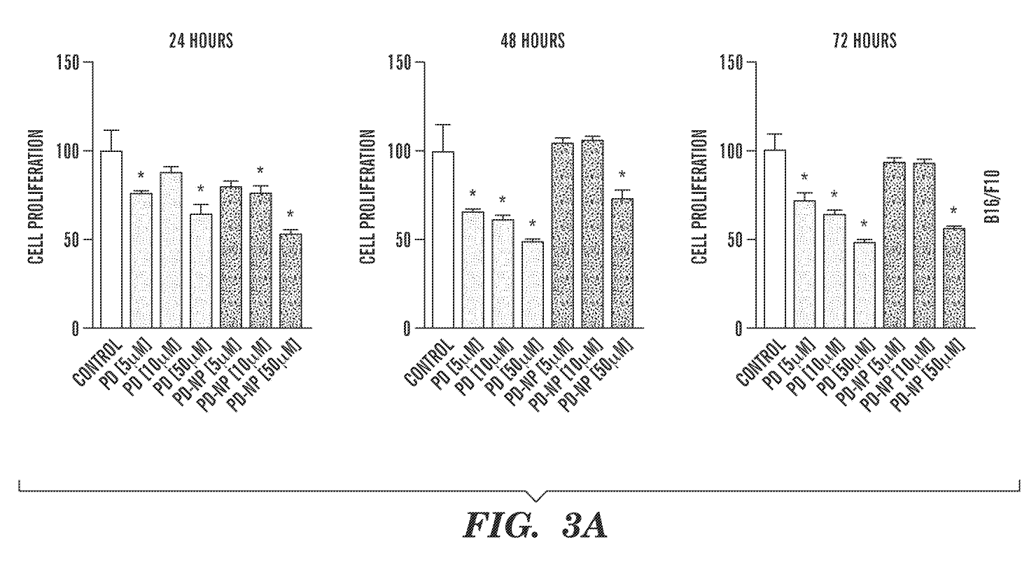

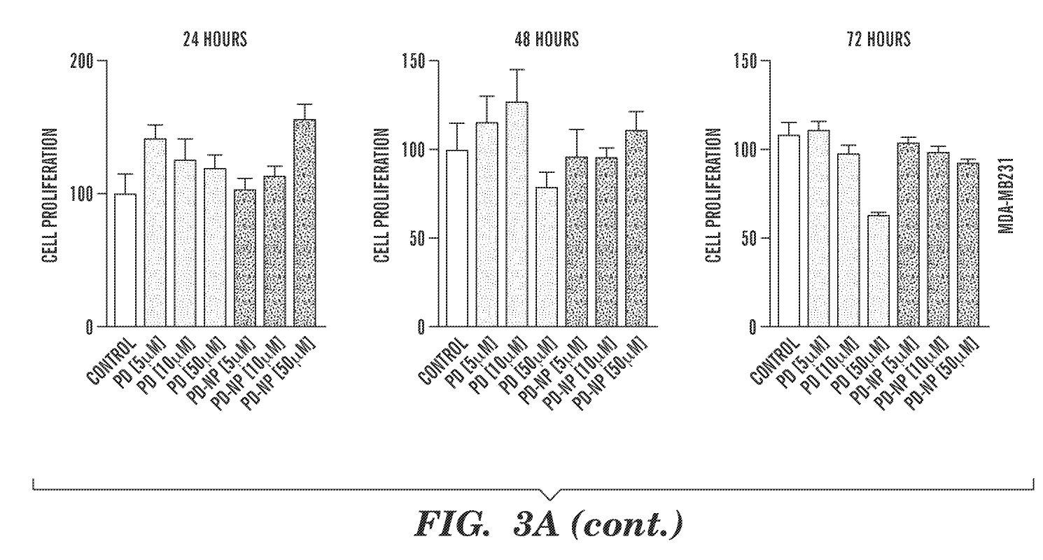

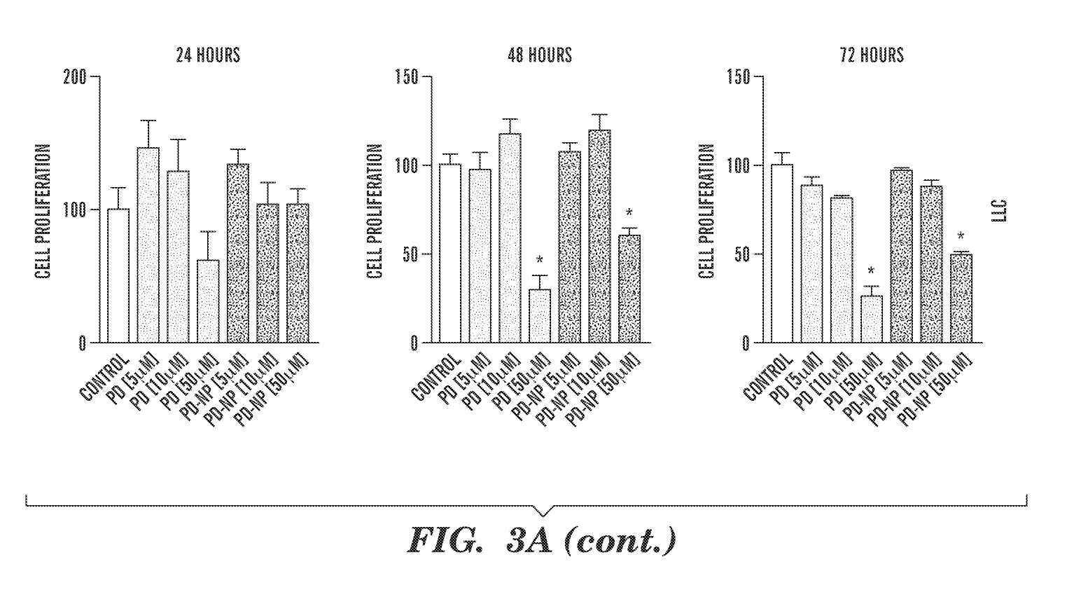

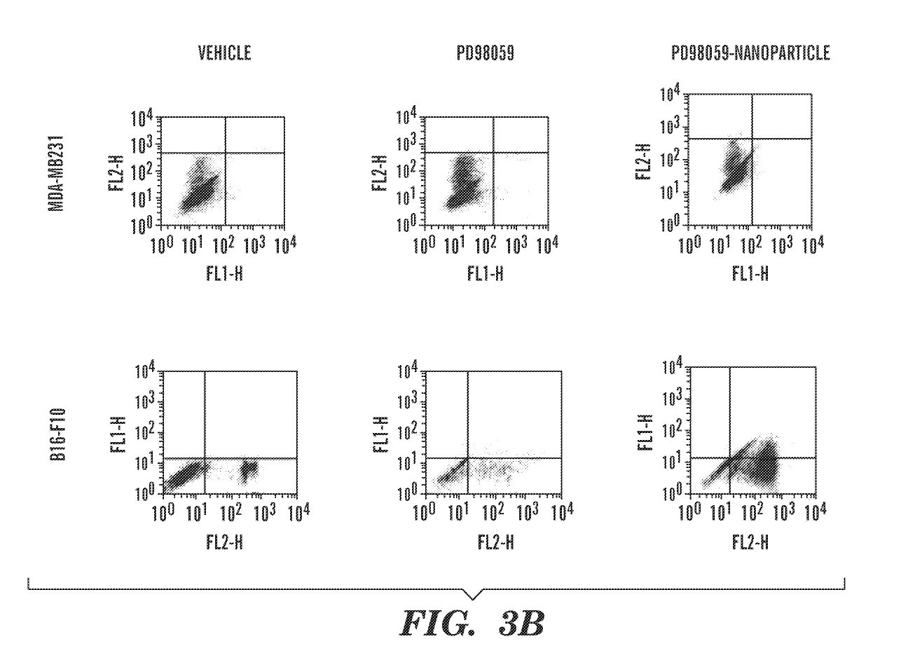

The inventors next evaluated the anticancer effects of the PD98059-nanoparticle as compared with free PD98059 in a series of in vitro cytotoxicity assays. They used three different cancer cell lines for this study, the B16/F10 melanoma cells, the MDA231 breast cancer cells and Lewis lung carcinoma cells. Western blot of the cell lysates revealed that although the phosphoERK1/ERK ratio was similar across the three cell lines, B16/F10 had an elevated level of ERK1, consistent with the fact that melanoma has elevated Ras signaling. The activated ERK, which is downstream of MEK signaling, confirmed that these were appropriate cells to study the effects of nanoparticle-mediated MAPK inhibition. The temporal release kinetics of the nanoparticle were also factored in, and cells incubated for different time periods. The cells were incubated for 24, 48 and 72 hrs of incubation in the presence of increasing concentrations of free drug or nanoparticles. The viability of the cells at the end of the incubation period was quantified using a colorimetric MTS assay. As shown in FIG. 3A, there was more cell kill at 24 hours with the free drug as compared with the PD98059-nanoparticle treatment, although this distinction was lost by 72 hours, thus confirming the temporal release control exerted by the nanoparticles. Furthermore, the inventors also observed different susceptibility to the nanoparticle-PD98059 between cancer cell lines. After 72 hrs of incubation with nanoparticles (50 μM PD98059) treatment, the proportion of live cell remaining were 92% for MDA-MB-231, 65% for LLC and 55% for B16-F10, indicating that MDA231 was resistant to PD98059-nanoparticle, while B16/F10 was most susceptible. The latter is consistent with the upregulated MAPK signaling in melanoma. Hence, for subsequent studies, focused was on the MDA-MB-231 and B16-F10 cell lines and using 50 μM of drugs.

To elucidate the mechanism underlying the effect on cell viability, the inventors studied whether the cells were undergoing apoptosis. MDA-MB-231 and B16-F10 were treated with PD98059-nanoparticle or free drug for 48 hours, and then labeled with Annexin V-FITC in conjunction with propidium iodide. As seen in FIG. 3B, the treatments failed to induce significant apoptosis in MDA321 cells. In contrast, the free drug and NP resulted in a 61-fold and a 360-fold increase, respectively, in late apoptosis in the B16-F10 cell line (FIG. 3B). This discrepancy between PD98059 and NP was accounted for by the fact the number of cells that were in the necrotic stage following free drug treatment was 51-fold higher than the cells treated with PD98059-nanoparticle, consistent with the slow and sustained release of the active agent from the nanoparticle.

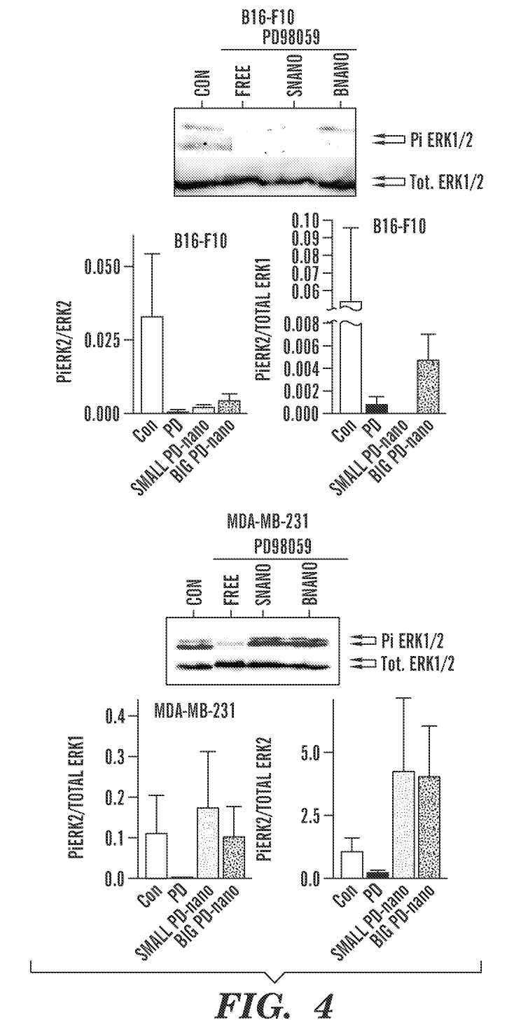

The inventors next evaluated the effect of the treatments on the phosphorylation status of ERK in the two cell lines. Both cell lines, MDA-MB-231 and B16-F10 were treated with 50 μM free PD98059 or as nanoparticles, and the cell lysates were subjected to immunoblotting against the phosphorylated and total forms of ERK1/2. In the case of MDA-MB-231, the free drug inhibited ERK signaling by 5-fold, whereas NP had no significant effect. In the case of B16-F10, however, both PD98059 and NPs almost completely abolished the phospho-ERK signal (FIG. 4A). The differences in the phosphorylation of ERK could explain the distinct outcomes in terms of cell viability for the two cell lines. Interestingly, in an earlier study, minor size differences of nanoparticles were reported to affect the biological outcome [19]. The inventors therefore tested the MEK inhibitory activity of nanoparticles at the two halves of the size distribution, i.e. <100 nm and >100 nm, observed no significant differences their efficacy to block ERK phosphorylation.

### Endocytosis and Intracellular Localization of Nanoparticles.

To track the uptake and distribution of the nanoparticles in the cells, the inventors engineered the nanoparticles with PLGA that was labeled with fluorescein (FITC). The uptake of FITC-NP into the B16-F10 cells was tracked at 15 and 30 min, as well as 2, 12 and 24 hrs. The lysosomal compartments of live cells were stained with LysoTracker (red) probe. Colocalization of the fluorescent signals from the nanoparticle and the lysosomes in the merged images (yellow) indicated that the FITC-nanoparticles were internalized into the lysosomes as early as 30 min in B16-F10 (data not shown).

### In Vivo Efficacy in B16/F10 Melanoma Model.

To validate the therapeutic efficacy of this treatment, the inventors randomly sorted mice bearing established B16/F10 melanomas into five groups, which received three doses of one of the following treatments (i) vehicle control (ii) free PD98 059 (5 mg/kg) (iii) PD98059-nanoparticle (equivalent to 5 mg/kg of PD98059) (iv) Cisplatin (1.25 mg/kg) and (v) PD98059-nanoparticle (equivalent to 5 mg/kg of PD98059)+Cisplatin (1.25 mg/kg). Cisplatin was administered intraperitoneally one day after the PD98059 administration in order to achieve a sequential biological effect of MAPK-inhibition followed by induction of chemotherapy-induced cytotoxicity. The mice injected with only vehicle formed large tumors by day 14, and consequently, were euthanized. The animals in the other groups were also sacrificed at the same time point to evaluate the effect of the treatments on tumor pathology.

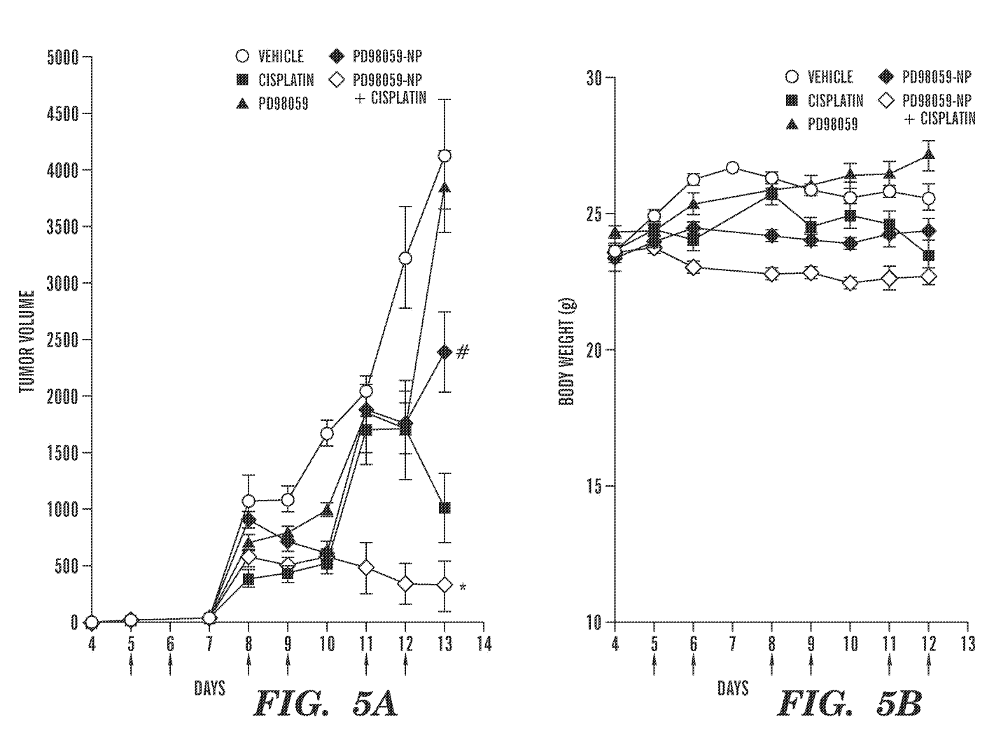

As shown in FIG. 5a, treatment with free PD98059 only partially inhibited tumor progression. In contrast, treatment with PD98059-nanoparticle resulted in a significant inhibition of tumor growth. Furthermore, pretreatment with PD98059-nanoparticles combined with cisplatin, exerted an antitumor effect that was significantly greater than either drug alone. This was consistent with gross pathological analysis of the tumor sections stained with hematoxylin-eosin that revealed large necrotic areas following treatment with the PD98059-nanoparticles, cisplatin, and a significant increase when the two treatments were combined together (data not shown). Greater than 10% loss of body weight following chemotherapy is an indication of non-specific toxicity [21]. As shown in FIG. 5b, none of the treatments induced any significant loss of body weight.

To dissect the mechanism of action for the increased antitumor activity of PD98059-nanoparticle and the synergism observed with cisplatin, tumor cross-sections were immunostained for phosphorylated ERK, which is downstream of PD98059-target, MEK. Phosphorylated ERK was detected in vehicle-treated tumors as well as those treated with free PD98059 or cisplatin alone (data not shown). In contrast, treatment with PD98059-nanoparticle induced significant inhibition of intratumoral ERK phosphorylation alone or when combined with cisplatin (data not shown). The inventors next evaluated the tumors for apoptosis using TUNEL-staining. Treatment with PD98059 alone had minimal effect but both PD98059-nanoparticle and cisplatin induced significant levels of apoptosis (data not shown). Furthermore, the apoptosis induced by cisplatin was potentiated by the pretreatment with PD98059-nanoparticle, which could explain the greater antitumor outcome seen as compared with either drug alone.

Herein, for the first time, is reported the use of a nanotechnology-based approach to target an oncogenic pathway. The inventors engineered nanoparticles from a PLGA-based polymer, which is well characterized and is approved by the FDA, but overcame the limitations of traditionally lower drug loadings with linear PLGA by developing a hexadentate variant of PLGA that amplified drug loading 20-fold. Furthermore, the inventors demonstrated that a compositional 1:5 ratio of PLGA-PEG and hexadentate-PLGA-(6)PD98059 results in nanoparticles of uniform and optimal size with efficient surface-coating with polyethylene glycol. The nanoparticle enabled a sustained-release of PD98059, which blocked the MAPK signaling cascade, and furthermore exerted a greater inhibition of tumor growth compared to free drug in a melanoma model. Excitingly, it potentiated the anticancer effect of cisplatin, a first line cytotoxic therapy for cancer, without inducing any additional gross toxicity, suggesting that nanodelivery of targeted therapeutics can emerge as a novel strategy for the management of cancers that are dependent on aberrant oncogenic pathways.

MEK1/2 are dual-specificity kinases that phosphorylate and activate ERK, the classical MAP kinase [2]. They lie downstream of RAS/RAF, which are the most commonly mutated members of the MAPK pathway. Additionally, MEK/ERK signaling is also activated downstream of growth factor signaling through kinase receptors, including epidermal growth factor receptor and MET receptor, which are implicated in tumorigenesis [21,22]. As a result, targeting MEK or ERK offers the possibility of exerting an antitumor effect even in the absence of RAS/RAF mutations. Activated ERK regulates the functions of multiple molecules that are implicated in cell cycle including p21^{Cip1}, p16^{Ink4a}, p15^{Ink4b}, and can additionally phosphorylate Bad, which contributes to its inactivation and sequestration by 14-3-3 proteins resulting in activation of Bc1-2 and an antiapoptotic response [23]. The inhibition of cell proliferation and the induction of apoptosis following treatment of the tumor cells with free PD98059 or PD98059-nanoparticle were consistent with the inhibition of phosphorylation of ERK, and the resultant blockage of these downstream proliferative and antiapoptotic signals. Interestingly, while incubation with cell lysates from all the three cell lines resulted in the release of the active agent from the nanoparticle, only B16/F10 melanoma and Lewis lung carcinoma cells were susceptible to the PD98059-nanoparticles, and there was no effect on the MDA-MB-231 breast cancer cells. This indicated that distinctions exist between tumor cells in their response to nanoparticles.

Interestingly, although the maximal effects of free PD98059 and as a nanoparticle were not differentiated in the in vitro assay, a significant improvement in the therapeutic efficacy in vivo with the nanoparticle-PD98059 was observed. This is consistent with earlier findings where nanoparticle-conjugated drug had greater activity in vivo, and arises from the preferential delivery of the nanoparticles to the tumor resulting in increased intratumoral concentrations as compared with free drug and the sustained release of the drug [24]. Indeed, an increased uptake of fluorescently-labeled pegylated nanoparticles in the tumor in vivo as compared with other tissues was observed [16]. This is further validated by the greater inhibition of intratumoral ERK signaling and resulting apoptosis when treated with the PD98059-nanoparticles as compared with the free drug. Excitingly, this mechanism-based induction of apoptosis translated into a synergistic effect when combined with a traditional chemotherapeutic agent, cisplatin, which induces apoptosis by intercalating with the DNA.

Several components of the present study can facilitate future therapy in humans: (i) optimization of loading of drugs using the hexadentate PLGA to engineer the nanoparticles allows the achievement of clinically-relevant doses, (ii) the increased in vivo efficacy of nanoparticle-MEK inhibitor as compared to the free drug indicates that nanoparticle could evolve as a powerful platform for targeting the oncogenic pathways, and (iii) the synergistic effects observed when PD98059-nanoparticles were combined with cisplatin indicates that this could evolve as a powerful multipronged strategy for the management of cancer. In summary, this study, for the first time, describes a nanotechnology-based strategy for targeting ‘targeted’ therapeutics to the tumor, which enhances antitumor outcomes and may emerge as a new paradigm in the management of cancers.

### REFERENCES

1. Anonymous. American Cancer Society. Cancer Facts and Figures 2008. Atlanta: American Cancer Society 2008.
2. Downward J. Targeting RAS signaling pathways in Cancer Chemotherapy. Nature Reviews Cancer. 2003; 3: 11-22
3. Davies H, Bignell G R, Cox C et al. Mutations of the BRAF gene in human cancer. Nature. 2002; 417: 949-54.
4. Friday B B, Adjei A A. Advances in targeting the Ras/Raf/MEK/Erk mitogen activated protein kinase cascade with MEK inhibitors for cancer therapy. Clin. Cancer Res. 2008; 14: 342-346.
5. Bos J L, Fearon E R, Hamilton S R, et al. Prevalence of ras gene mutation in human colorectal cancers. Nature. 1987; 327: 293-297.
6. Brose M S, Volpe P, Feldman M, et al. BRAF and RAS mutations in human lung cancer and melanoma. Cancer Res. 2002; 62: 6997-7000.
7. Sieben N L, Macropoulos P, Roemen G M, et al. In ovarian neoplasms, BRAF but not KRAS mutations are restricted to low-grade serous tumors. J. Pathol. 2004; 202: 336-40.
8. Peer D, Karp J M, Hong S, et al. Nanocarriers as an emerging platform for cancer therapy. Nature Nanotechnology. 2007; 2, 751-760
9. Zhang L, Gu F X, Chan J M, et al. Nanoparticles in medicine: therapeutic applications and developments. Clin. Pharmacol. Ther. 2008; 83: 761-9.
10. Moghimi S M, Hunter C A, Murray C J. Long-circulating and target-specific nanoparticles: theory to practice. Pharmacol. Rev. 2001; 53: 283-318
11. Allen T M, Hansen C, Martin F, Redemann C, Yau-Young A. Liposomes containing synthetic lipid derivatives of poly(ethylene glycol) show prolonged circulation half-lives in vivo. Biochim Biophys Acta. 1066:29 (1991).
12. Yuan F, Leunig M, Huang S K, Berk D A, Papahadjopoulos D, Jain R K. Microvascular permeability and interstitial penetration of sterically stabilized (stealth) liposomes in a human tumor xenograft. Cancer Res. 54:3352 (1994).
13. Boulikas T, Stathopoulos G P, Volakakis N, Vougiouka M. Systemic Lipoplatin infusion results in preferential tumor uptake in human studies. Anticancer Res. 2005; 25: 3031-9.
14. Dudley D T, Pang L, Decker S J, Bridges A J, Saltiel A R. A synthetic inhibitor of the mitogen-activated protein kinase cascade. Proc. Natl. Acad. Sci. USA. 1995; 92: 7686-7689.
15. Alessi D R, Cuenda A, Cohen P, Dudley D T, Saltiel A R. PD098059 is a specific inhibitor of the activation of mitogen-activated protein kinase kinase in vitro and in vivo. J. Biol. Chem.
1995; 270: 2 7489-94.
16. Sengupta, S., Eavarone, D., Capila, I., Zhao, G., Watson, N., Kiziltepe, T., Sasisekharan, R. (2005) Temporal targeting of tumor cells and neovasculature with a nanoscale delivery system. Nature 436: 568-572.
17. Mosqueira V C, Legrand P, Gulik A, Bourdon O, Gref R, Labarre D, Barratt G. Relationship between complement activation, cellular uptake and surface physicochemical aspects of novel PEG-modified nanocapsules. Biomaterials. 2001; 22: 2967-79.
18. Fishburn C S. The pharmacology of PEGylation: balancing PD with PK to generate novel therapeutics. J. Pharm. Sci. 2008; 97: 4176-4183.
19. Gaumet M, Vargas A, Gurny R, Delie F. Nanoparticles for drug delivery: the need for precision in reporting particle size parameters. Eur. J. Pramaceutics. Biopharmaceutics. 2008; 69: 1-9.
20. Clark D L, Andrews P A, Smith D D, et al. Predictive value of preclinical toxicology studies or platinum anticancer drugs. Clin. Cancer. Res. 1999; 5: 1161-1167.
21. Marks J L, Gong Y, Chitale D, et al. Novel MEK1 mutation identified by mutational analysis of epidermal growth factor receptor signaling pathway genes in lung adenocarcinoma. Cancer Res. 2008 Jul. 15; 68(14):5524-8
22. Sengupta S, Sellers L A, Li R C, Gherardi E, Zhao G, Watson N, Sasisekharan R, Fan T P. Targeting of mitogen-activated protein kinases and phosphatidylinositol 3 kinase inhibits hepatocyte growth factor/scatter factor-induced angiogenesis. Circulation. 2003 Jun. 17; 107(23):2955-61.
23. McCubrey J A, Steelman L S, Chappell W H, et al. Roles of the Raf/MEK/ERK pathway in cell growth, malignant transformation and drug resistance. Biochemica et Biophysica Acta. 2007; 1773: 1263-1284.
24. Tang N, Du G, Wang N, et al. Improving penetration in tumors with nanoassemblies of phospholipids and doxorubicin. J. Natl. Cancer Inst. 2007; 99:1004-15.

### Example 22

### LY29402-Entrapped Nanoparticles

### Materials:

All the solvents were purchased from Aldrich, Fluka and Fisher unless otherwise stated and used without any further purification. The poly (lactic-co-glycolic acid) (Mw˜66 kDa) having a lactic/glycolic molar ratio of 50/50 was procured from Lakeshore Chemicals. LY294002 was purchased from Tocris. UV-VIS spectra were measured using Shimadzu UV-2450 UV-VIS Spectrophotometer. Malvern Nanozetasizer was used to measure Dynamic Light scattering. TEM was measured by Jeol E M. CellTiter 96 AQueous One Solution Cell Proliferation (MTS) Assay was obtained from Promega Corporation (Madison, Wis.). AnnexinV-Alexa Fluor 488, the LysoTracker Red probe and the QTracker Red cell labeling kit were all from Invitrogen (Carlsbad, Calif.). Polyclonal antibodies specific for actin, as well as for the phosphorylated (pi-AKT) and total form (AKT) of AKT were purchased from Cell Signaling Technology (Danvers, Mass.). Fibroblast growth factor (FGF) and vascular endothelial cell growth factor (VEGF) were from R&D Systems (Minneapolis, Minn.). Matrigel basement membrane matrix was obtained from BD Biosciences (San Jose, Calif.).

### Synthesis of LY294002-Encapsulated Nanoparticle (NP-LY):

Nanoparticles (NP) were formulated using an emulsion-solvent evaporation technique. 50 mg PLGA was dissolved completely in 2.5 mL acetone and mixed with 3 mg of LY294002 (dissolved in 0.5 mL methanol). The entire solution was emulsified into 25 mL of 2% aqueous solution of PVA (80% hydrolyzed, Mw˜9000-10,000) by slow injection with constant homogenization using a tissue homogenizer. This mini emulsion was added to a 100 mL 0.2% aqueous solution of PVA (80% hydrolyzed, Mw˜9000-10,000) with rapid stirring for 4 h at room temperature to evaporate any residual acetone or methanol. NP size fraction was recovered by ultracentrifugation at 20,000 and 80,000×g and the NPs were lyophilized for 24 h. Sizing was performed by dynamic light scattering (DLS) and transmission electron microscopy (TEM). The NPs were washed thoroughly with double distilled water to remove excess PVA before preparing the sample for TEM. The LY294002 loading in the NPs was determined by UV-VIS spectroscopy at the wavelength λ=298 nm.

### Transmission Electron Microscopy (TEM) of the Nanoparticles:

The NP were fixed in 2.5% gluteraldehyde, 3% paraformaldehyde with 5% sucrose in 0.1M sodium cacodylate buffer (pH=7.4), embedded in low temperature agarose and post fixed in 1% OsO4 in veronal-acetate buffer. The sample was stained in block overnight with 0.5% uranyl acetate in veronal-acetate buffer (pH=6.0) then dehydrated and embedded in epon-812 resin. Sections were cut on a Leica ultra cut UCT at a thickness of 70 nm using a diamond knife, stained with 2.0% uranyl acetate followed by 0.1% lead citrate and examined using a Philips EM410.

### Physicochemical Release Kinetics Characterization:

LY294002-encapsulated NP were suspended in 500 μL of PBS and sealed in a dialysis bag (MWCO˜1000 Da). The dialysis bag was incubated in 1 mL of PBS buffer at room temperature with gentle shaking. 10 μL of aliquot was extracted from the incubation medium at predetermined time intervals, dissolved in 90 μL DMF and released LY294002 was quantified by UV-VIS spectroscopy at characteristic wavelength of LY294002, λ=298 nm. After withdrawing each aliquot the incubation medium was replenished by 10 μL of fresh PBS.

### Cell Culture:

Cancer cells were obtained from American Type Tissue Culture Collection (Rockville, Md.) and were maintained in DMEM supplemented with 10% FBS and antibiotic/antimycotic (all from Invitrogen). MDA-MB-231 is a human breast human adenocarcinoma cell line whereas B16-F10 and LLC are derived from mouse melanoma and Lewis lung carcinomas models, respectively. All cells were grown on 100 mm dishes and subcultured using trypsin (0.25%) and EDTA (0.01%) treatment and replated at different ratios depending on the experiment. Cells were switched to 1% serum prior to drug addition, in order to quantitative the effects of the drug proper.

Human umbilical vein endothelial cells (HUVEC) were obtained from Cambrex Bio Science (Hopkinton, Mass.) and cultured in EGM-2 medium according to the manufacturer's protocol. A single donor was obtained at passage one, and cells were used between passages 2 and 5. After reaching the first confluence in 100 mm dishes (within 6 to 7 days), the cells were subcultured following trypsin (0.025%) and EDTA (0.01%) application, and plated at various densities depending on the experiment. For all experiments, HUVEC were synchronized overnight using serum reduced medium (0.1% FBS) prior to drug addition, except in the case of the tube assay. For the MTS assay and immunoblotting, HUVEC were also treated with 5 nM of FGF or VEGF, respectively.

For the Zebrafish assays, MDA-MB-23 1 stably expressing GFP (MDA-MB-23 1/GFP) were generated using the pAcGFP-C1 expression plasmid (Clontech, Mountain View, Calif.). This vector contains a codon-optimized GFP gene which yields maximal expression and prolonged fluorescence in mammalian cells, as well as a gene coding for neomycin resistance, thus allowing for geneticin (G41 8) selection. 1.5×10⁵ MDA-MB-231 cells were seeded in 6-well plates overnight and then transfected with 2 ug pAcGFP1-C1 using Lipofectin reagent (Invitrogen) for 24 hours. Cells allowed to recover for 24 hrs, after which time fresh growth medium supplemented with 1 mg/ml geneticin was added until drug-resistant colonies appeared. These drug resistant cells were then propagated and sorted based on the top 5% of fluoresence using a MoFlo3 cell sorter (The David H. Koch Institute for Integrative cancer Research, Cambridge, Mass.).

The drugs used throughout experiments consisted of the free drug, LY294002 (LY) or LY294002-encapsulated nanoparticles (NP-LY).

### MTS Cytotoxicity Assay:

Cancer or endothelial cells in 96-well plates were incubated with various doses of free LY or NP-LY for 24, 48 and 72 hrs. The percentage of viable cells were then quantified with 3-(4,5-Dimethylthiazol-2-yl)-5-(3-carboxymethoxyphenyl)-2-(4-sulfophenyl)-2H-tetrazolium (MTS) from the CellTiter 96 AQueous One Solution kit. MTS is reduced by mitochondrial dehydrogenases of live cells, yielding a colored adduct that can be read spectrophotometrically. Briefly, the cells were washed with PBS, incubated with 0.3 mg/ml of MTS, in basal medium without phenol red, for 4 hrs at 37° C. and absorbance was then measured at 490 nm in a plate reader (Versamax, Molecular Devices, Sunnyvale, Calif.). Final absorbance, corresponding to cell proliferation, was plotted after removing background values from each data point.

### AnnexinV-FITC Apoptosis Study:

Cancer cells grown in 6-well plates were treated with drugs for 48 h, and incubated with 5 μl AnnexinV-Alexa Fluor 488 in binding buffer (10 mM HEPES, 140 mM NaCl, 2.5 mM CaCl₂, pH 7.4) for 15 min in the dark, according to the manufacturer's protocol. Cells were then washed with binding buffer, counterstained with propidium iodide and immediately processed for FITC and propidium iodide staining using a Becton Dickinson FACSCalibur flow cytometer (excitation 488 and 585 nm, respectively). AnnexinV-Alexa Fluor 488, propidium iodide or both were omitted for the negative controls.

### Drug Uptake and Metabolism:

LysoTracker probes are weakly basic amines which accumulate in the acidic compartments of live cells and can hence be used to track drug uptake and metabolism. MDA-MB-231, B16-F10 and HUVEC cells were seeded on glass coverslips in 24-well plates until subconfluency, and then treated with 5.6 mg/ml FITC-conjugated nanoparticles (FITC-NP) for a time-course ranging from 30 min to 24 hrs. At the indicated times, cells were washed twice in PBS and incubated in LysoTracker Red (Ex: 577 nm; Em: 590 nm) for 30 min at 37° C. Cells were then washed again, fixed in 4% paraformaldehyde and mounted using Prolong Gold antifade reagent (Invitrogen). Images taken in 3 random fields were captured at 20× and 40× using an inverted microscope (Nikon Eclipse, Melville, N.Y.) equipped with blue and green filters in order to visualize FITC-NP and LysoTracker Red fluorescence, respectively. Cells incubated either only FITC-NP or Lysotracker red served as negative controls.

### Immunoblotting:

Cancer or endothelial cells were washed twice with PBS and directly lysed in 3× loading buffer containing 12% sodium dodecyl sulfate, 15% 2-mercaptoethanol, 1 mM sodium orthovandate and protease inhibitor cocktail tablets from Roche Applied Science (Indianapolis, Ind.). Cells were further homogenized by passing the lysates 3 times through an insulin needle. Samples were then heated for 5 min at 100° C. and equal amounts loaded onto tris-glycine SDS-polyacrylamide gels. Proteins were electrophoretically transferred onto polyvinylidene difluoride membranes, blocked for 1 h with 7% non-fat dry milk, and subsequently incubated overnight at 4° C. with primary antibodies directed against the phosphorylated or total forms of AKT. Proteins were detected with horseradish peroxidase-conjugated anti-rabbit secondary antibodies and Lumi-LightPLUS Western Blotting Substrate (Roche Applied Science). The blots were developed using GeneSnap and optical densities off the protein bands quantified using GeneTools (both from SynGene, Frederick, Md.). Predetermined molecular weight standards were used as markers. Proteins were normalized against actin.

### HUVEC Tube Assay:

The effect of the drugs using an in vitro angiogenic assay were quantified as follows. HUVEC were seeded in 24-well plates, on glass coverslips that had been coated with matrigel, in medium containing 50 μM of LY or NP-LY for 24 and 48 hrs. At each time points, cells were fixed with 4% paraformaldehyde and immediately visualized under inverted light microscopy at 20× magnification (Nikon Eclipse). Since the matrigel tube assay often yields high variability, we took 10 fields of view per coverslip with 5 coverslips per conditions, and furthermore, used three morphometric methods using the NIS-elements software (Nikon, Melville, N.Y., courtesy of Dr Jeffrey M. Karp) to quantify the results. In the first such method, we measured the length of each tube per field, in the second we measured the associated number of nodes, whereas in the third method, we used a standard graticule to measure the number of vessels falling on each intersection.

### Zebrafish Xenograft Assay:

In order to assess the effect of NP-LY on in vivo angiogenesis, we used the transparent Danio Rerio (zebrafish) model. Zebrafish [TubingenAB and tg(Fli:GFP)] embryos were maintained at 28° C. in standard E3 solution buffered with 2 mM HEPES. 48 hrs post-fertilization (hpf) embryos were anesthetized with 0.04 mg/ml of Tricaine and were dechlorinated manually. Embryos were injected in the yolk sac, near the subintestinal vessels, with around 1000 cells resuspended in matrigel, in the presence or absence of NP-LY and with a constant volume of 9.2 nL using a Nanoject II (Drummond Scientific), based on the protocol of Stefania et al. [3726]. The cells used for the experiments were either MDA-MB-231/GFP or B16-F10 labeled with the QTracker Red kit, according to the manufacturer's protocol. Images were taken both in real-time and after alkaline phosphatase staining using nitroblue tetrazolium chloride and 5-Bromo-4-chloro-3-indolyl phosphate, toluidine salt (Roche, Nutley, N.J.), in order to visualize their vasculature. Brightfield and fluorescence imaging of the embryos was performed with a Nikon SMZ1500 stereomicroscope and SPOT Flex camera. Image sequences were obtained with the same set-up and exported as movies to match live flow patterns. All procedures were approved by Harvard University IACUC.

### Results and Discussion

One of the best characterized oncogenic signal transduction cascades is the phosphatidylinositol 3-kinase (PI3K)-Akt pathway, which is deregulated in a majority of tumors [6]. PI3K is generally recruited downstream of activated receptor tyrosine kinases, G-protein-coupled receptors or integrins at the plasma membrane, where it catalyzes the addition of a phosphate group at the 3′-position of the inositol ring of phosphoinositide/phosphatidylinositol (PI), which binds to the pleckstrin-homology domain of multiple proteins [7]. Activating mutations of the gene that encodes the catalytic subunit of class 1A PI3K have been implicated in ovarian and lung tumors [8, 9]. Similarly, phosphatase PTEN, which deactivates PI3K, has been shown to be mutationally or post-translationally inactivated or inhibited in other tumors, such as in glioblastoma, breast, melanoma, lung, hepatocellular carcinoma [10, 11, 12, 13, 14]. Furthermore, PI3K signaling has been implicated in tumor angiogenesis downstream of growth factors such as vascular endothelial growth factor and hepatocyte growth factor [15,16]. Thus inhibition of PI3K holds the promise of a multi-pronged strategy for tumor inhibition. We targeted this key oncogenic signaling pathway using a polymeric nanoparticle. The best-characterized PI3K inhibitor, 2-(4-morpholinyl)-8-phenylchromone (LY294002) is a selective and potent inhibitor which prevents PI3K-induced activation of AKT by competitively binding the ATP-binding pocket of PI3K's catalytic domain (IC₅₀=1.40 μM), resulting in a potent anti-tumor activity in vivo [17]. We used LY294002 (LY) as the model PI3K inhibitor for encapsulation in the nanoparticles (NP), which were engineered from biodegradable polylactic acid-glycolic acid (PLGA) co-polymers.

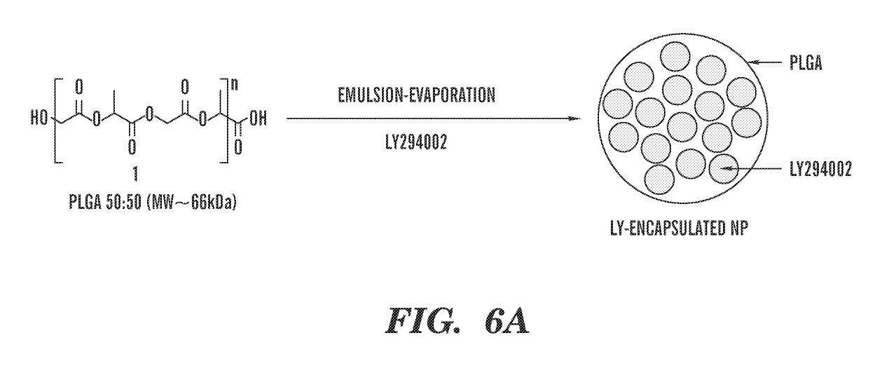

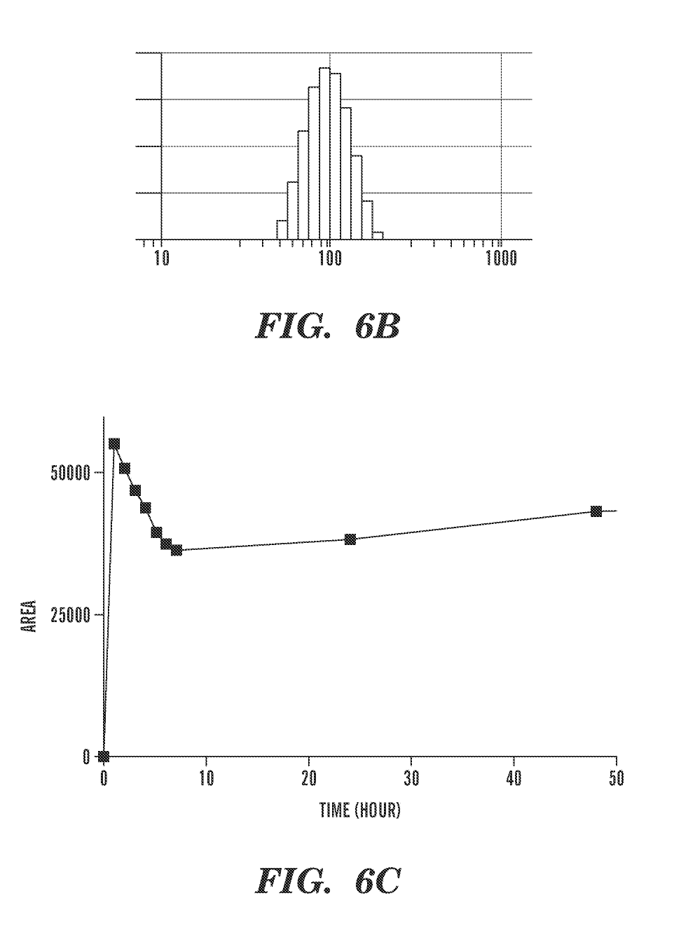

LY294002-entrapped nanoparticles (NP-LY) were synthesized using an emulsion-solvent evaporation technique (FIG. 6A). A PLGA/LY294002 mixture in acetone and methanol was emulsified into a 2% aqueous solution of PVA (80% hydrolyzed, Mw-9000-10,000) by slow injection with constant homogenization using a tissue homogenizer. This mini emulsion was added to a 0.2% aqueous solution of PVA (80% hydrolyzed, Mw 9000-10,000) with rapid mixing for 4 h at room temperature to evaporate any residual acetone or methanol. Nanoparticles were recovered by ultracentrifugation at 80,000×g, following which they were lyophilized for 24 h. The loading efficiency of LY294002 in the nanoparticle was 15% as determined by UV-VIS spectroscopy at the characteristic wavelength of LY294002, λ=298 nm. The surface morphology and size distribution of the nanoparticles were evaluated by transmission electron microscopy (TEM) and dynamic light scattering (DLS) experiments.

To prepare the nanoparticles for TEM, the nanoparticles were fixed in 2.5% gluteraldehyde, 3% paraformaldehyde with 5% sucrose in 0.1M sodium cacodylate buffer (pH=7.4), embedded the fixed nanoparticles in low temperature agarose, and post fixed in 1% OsO₄ in veronal-acetate buffer. The sample was stained in block overnight with 0.5% uranyl acetate in veronal-acetate buffer (pH=6.0), then dehydrated and embedded in epon-812 resin. Sections were cut on a Leica ultra cut UCT at a thickness of 70 nm using a diamond knife, stained with 2.0% uranyl acetate followed by 0.1% lead citrate and examined using a Philips EM410. The size distribution of the nanoparticles was found to be in the range 60-120 nm in diameter, which was confirmed from DLS measurements (FIG. 6B). It is well documented that nanoparticles in the optimal size range of 60-180 nm preferentially home into tumors by avoiding the reticuloendothelial system[18].

The release kinetics of LY294002 from the nanoparticles were studied next. LY294002-encapsulated nanoparticles were suspended in 500 μL of PBS and sealed in a dialysis bag (MWCO˜1000 Da). The dialysis bag was incubated in 1 mL of PBS buffer at room temperature with gentle shaking. 10 μL of aliquot was extracted from the incubation medium at predetermined time intervals, dissolved in 90 μL DMF and released LY294002 was quantified by reverse phase HPLC (Waters 2696 Separation Module) using Atlantis® dC18 5 μm column (4.6×150 mm) and Waters 486 Tunable Absorbance Detector at characteristic wavelength of LY294002, λ=298 nm using acetonitrile:water (80:20) as mobile phase at retention time t=4.8 min. As shown in FIG. 6C, the highest amount of LY294002 was released within 1 h of incubation, confirming the characteristic burst release profile that is associated with nanoparticles; it then started to decay and became saturated after 10 h, with sustained release for up to 150 hours.

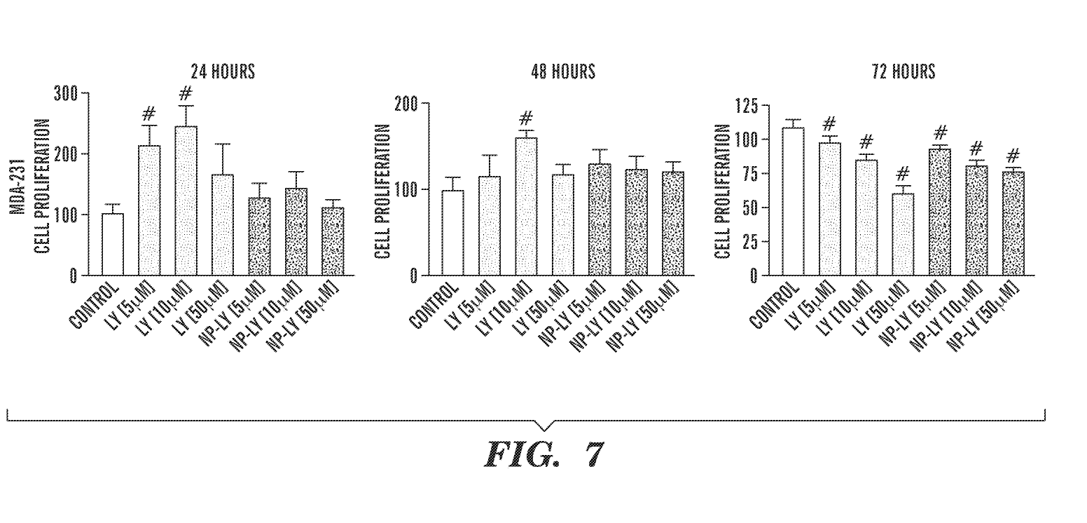

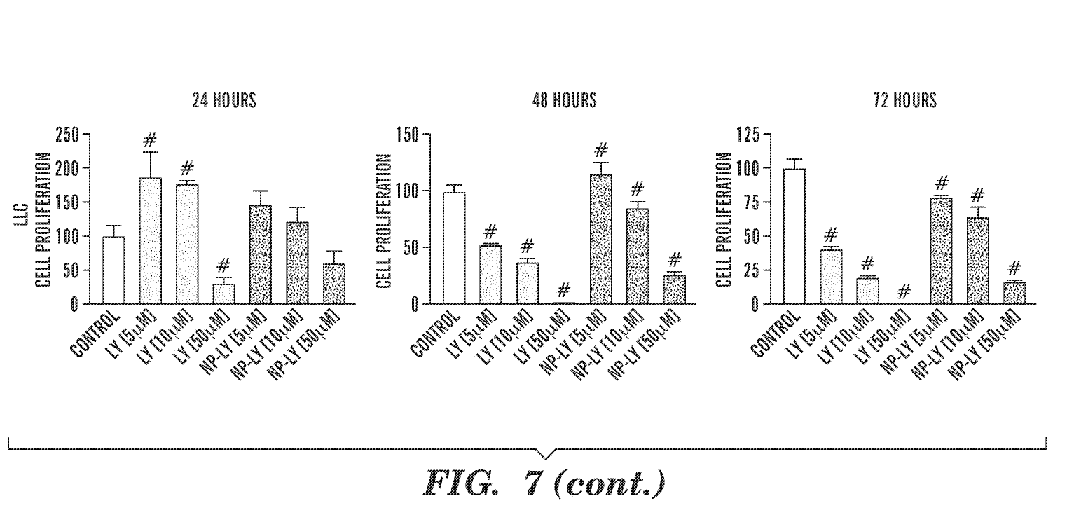

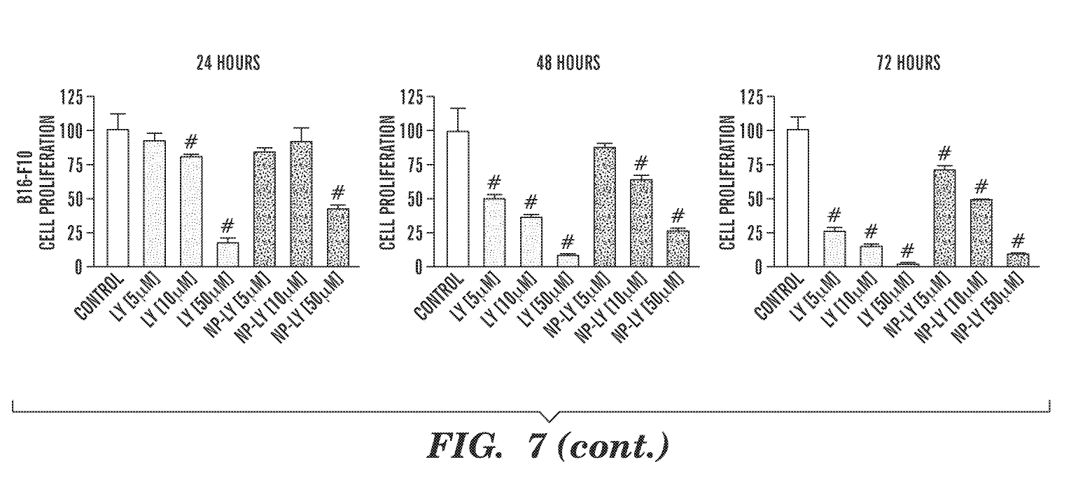

To measure cytotoxicity of NP-LY versus the free drug, a panel of three cancer cell lines was incubated with free LY294002 or NP-LY for 24, 48 and 72 hrs, following which the metabolic activity of these cells was measured using 3-(4,5-Dimethylthiazol-2-yl)-5-(3-carboxymethoxyphenyl)-2-(4-sulfophenyl)-2H-tetrazolium (MTS). As shown in FIG. 7, a slower onset of cytotoxic effect was observed following NP-LY-treatment as compared with the free drug (LY294002), thus confirming the temporal control over the release exerted by encapsulating LY294002 in the nanoparticles. Interestingly, different cytoxic responses between the cancer cell lines in response to both free LY294002 and NP-LY were observed. Significant cytotoxic effect on MD-MB-231 breast adenocarcinoma cells was apparent only at the higher concentrations and following 72 hrs of incubation with either the free or encapsulated drug. In contrast, both free LY294002 and NP-LY significantly inhibited the viability of Lewis lung carcinoma (LLC) cells and B16-F10 melanoma. For example, following 48 hrs of incubation with 50 μM of LY294002 or NP-LY, the proportion of viable cells as percentage of vehicle-treated control were 116% and 122% for MDA-MB-231, 2% and 25% for LLC and 8% and 27% for B16-F10 respectively, suggesting that MDA-MB-231 was refractory while both B16-F10 and LLCs were sensitive to the treatments with LY294002 or NP-LY. For further studies, B16/F10 and MDA-MB-231 cell lines were selected as examples of susceptible and refractory cells respectively.

To elucidate the differential sensitivity between both cell lines to LY294002 and NP-LY, the mechanisms of NP uptake and metabolism were evaluated using the LysoTracker Red probe. Nanoparticles from fluorescein-labeled PLGA were engineered for this study. To synthesize FITC-conjugated PLGA, PLGA (50 mg) was dissolved in 750 μL dichloromethane. NHS and EDC were added into the reaction mixture and stirred at room temperature for 2 h. FITC was dissolved in a mixture of dichloromethane and pyridine. FITC solution in pyridine was added into the activated PLGA solution and the reaction mixture was stirred at 4° C. for 24 h in dark. The reaction was diluted with DCM and quenched with 0.1 N HCl solution. The organic layer was extracted with DCM, washed with water, brine and dried over anhydrous sodium sulfate. The organic layer was filtered and evaporated to obtain the crude product. The PLGA-FITC conjugate was precipitated out from the crude product by addition of diethyl ether (40 mL). The polymer was centrifuged at 3220×g for 30 minutes. The supernatant was discarded and the polymer was washed thoroughly by diethyl ether and dried under vacuum overnight.

Breast adenocarcinoma (MDA-MB-231) and melanoma cells (B16-F10) were seeded on glass coverslips in 24-well plates and incubated with FITC-labelled nanoparticles f for 15 and 30 min, 2 h, 6 h, 12 h and 24 hrs. At indicated time points, lysosomal compartments of live cells were then stained with LysoTracker Red probe, and visualized using epifluorescence microscopy (40×) such that the merged images would appear yellow if the green FITC-NP were internalized into the red lysosomes. At least three independent measurements were performed per time-point. Nanoparticles were taken up by the B16-F10 cells earlier as compared with MDA-MB-231, with significant internalization occurring as early as 15 min in B16-F10, whereas comparable internalization was only observed after 6 hrs in MDA-MB-231 cells (data not shown). Furthermore, the colocalization of the FITC-NPs and the LysoTracker Red signals indicated that the nanoparticles were internalized into the lysosomes. The intracellular concentration of FITC-NP was observed to decrease by 12 hrs in MDA-MB-231 cells, as compared to 24 hrs in B16-F10 cells, demonstrating that the rate of drug clearance was significantly faster in the MDA-MB-231 cell line (data not shown). Drug refractoriness in MDA-MB-231 has been correlated with the over-expression of ATP-binding cassette (ABC) transporters [19]. This mechanism may underlie NP-LY clearance and could explain the limited efficacy seen with LY294002 in MDA-MB-231.

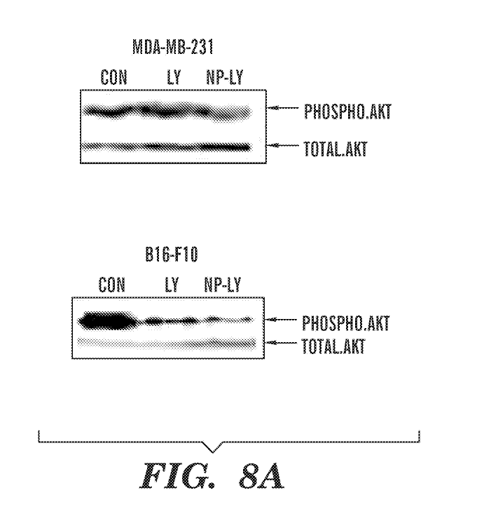

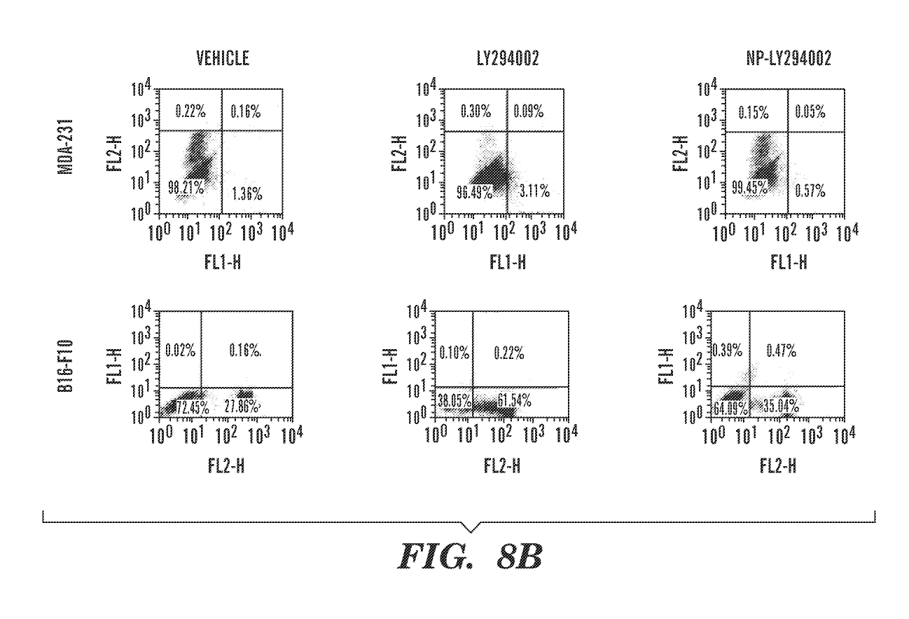

To further evaluate the underlying mechanisms of action of nanoparticles in cancer cells, MDA-MB-231 and B16-F10 were treated with 50 μM of LY294002 or NP-LY and subjected to immunoblotting against the phospho- and total forms of AKT (FIG. 8A). It is now well established that the activation of PI3K results in the generation of PIP3 on the inner leaflet of the plasma membrane, which recruits AKT by direct interaction with its PH domain [20]. At the membrane a serine/threonine kinase, PDK1, phosphorylates AKT on Thr308, which activates AKT. A second phosphorylation at Ser473 increases the activity. Indeed, AKT was found to be phosphorylated in both MDA-MB-231 and B16/F10 melanoma cells. Interestingly, whereas treatment with LY294002 or NP-LY had only minimal effect on inhibiting the phosphorylation of AKT in MDA-MB-231 cells, both free LY294002 and NP-LY inhibited AKT signaling in B16-F10 cells by up to 7-fold. Together with the uptake studies, this differential inhibition of AKT signaling could potentially explains the distinct sensitivities of MDA-MB-231 and B16-F10 with respect to both free and encapsulated LY294002.

One of the key biological consequences of activated AKT signaling is the inhibition of apoptosis through phosphorylation of several components of the cell death machinery. For example, AKT-mediated phosphorylation of BAD, a pro-apoptotic member of the BLC2 family of proteins, prevents its non-functional hereterodimerization with the survival factor BCL-X_{L}, leading to restoration of the anti-apoptotic function of BCL-X_{L} [21]. Furthermore, AKT-induced phosphorylation can inhibit the catalytic activity of pro-apoptotic caspase-9 [22], and also prevent the nuclear translocation of FKHR, a member of the Forkhead family of transcription factors, resulting in inactivation of FKHR gene targets including pro-apoptotic proteins such as BIM and FAS ligands [23]. To determine whether the LY294002 and NP-LY-induced inhibition of PI3K and subsequent block of AKT activation results in apoptosis of tumor cells, the cells were labeled with AnnexinV (conjugated to FITC), which binds to phosphatidylserine expressed on cell surface during apoptosis. Simultaneous staining of the cells with the vital dye, propidium iodide (PI), allowed the inventors to get a profile of early (AnnexinV-FITC+ve, PI−ve) versus late apoptosis (AnnexinV-FITC+ve; PI+ve) (FIG. 8C). Fluorescence activated cell sorting analysis following annexinV-FITC and PI staining revealed that only 3.11% and 0.09% of the MDA-MB-231 cells were in early and late apoptosis following LY294002 treatment. Similarly, treatment with NP-LY resulted in 0.57% and 0.05% of the MDA-MB-231 cells in early and late apoptosis phases respectively. In contrast, treatment of B16/F10 melanoma cells with LY resulted in 61.64% and 0.22% of the cells shifting to early and late apoptosis respectively. Similarly, treatment with NP-LY resulted in 35.04% and 0.47% of the cells shifting to early and late apoptosis, consistent with the temporal nature release of LY294002 from the nanoparticles. The levels of apoptosis in MDA-MB-231 breast cancer and B16/F10 melanoma cells correlated well with the levels of phosphorylated AKT following different treatments, indicating that the cytotoxic effects were mediated through an AKT-dependent apoptotic mechanism. Intriguingly, the failure of LY294002 or NP-LY to induce apoptosis in the MDA-MB-231 cell line despite the basal activated state of AKT in this cell line is not entirely unexpected. While it may arise from reduced uptake or increased efflux of the active agent or the nanoparticle inside the cells as observed in this study, it may also arise from compensatory mechanisms that are simultaneously activated in cancer cells [24,25]. For example, in an elegant study, Rosen et al demonstrated that induction of PTEN (thereby downregulation of PI3K signaling) and inhibition of epidermal growth factor receptor induced a synergistic apoptosis response [26], by blocking distinct pathways that independently converge into phosphorylation of the pro-apoptotic protein BAD at two distinct sites [26]. Indeed, in another study, a combination of a AKT/mammalian target of rapamycin (mTOR) inhibitor and a MAPK kinase 1 inhibitor was shown to dramatically impact tumor progression in a hormone-refractory prostate cancer model [27]. These studies indicate that tumor progression can depend on multiple independent signaling pathways, and any broad nanoparticle-based strategy for targeting tumor cells will potentially require the inhibition of multiple targets besides PI3K. Interestingly, the compositions described herein can be used for inhibiting multiple signal transduction targets, and with preliminary results indicating that inhibitors of MAPK and PI3K can synergize in the case of MDA-MB231.

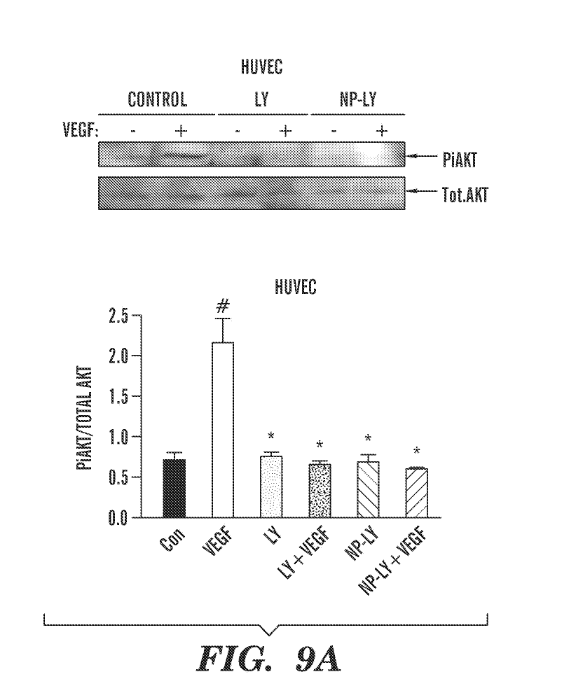

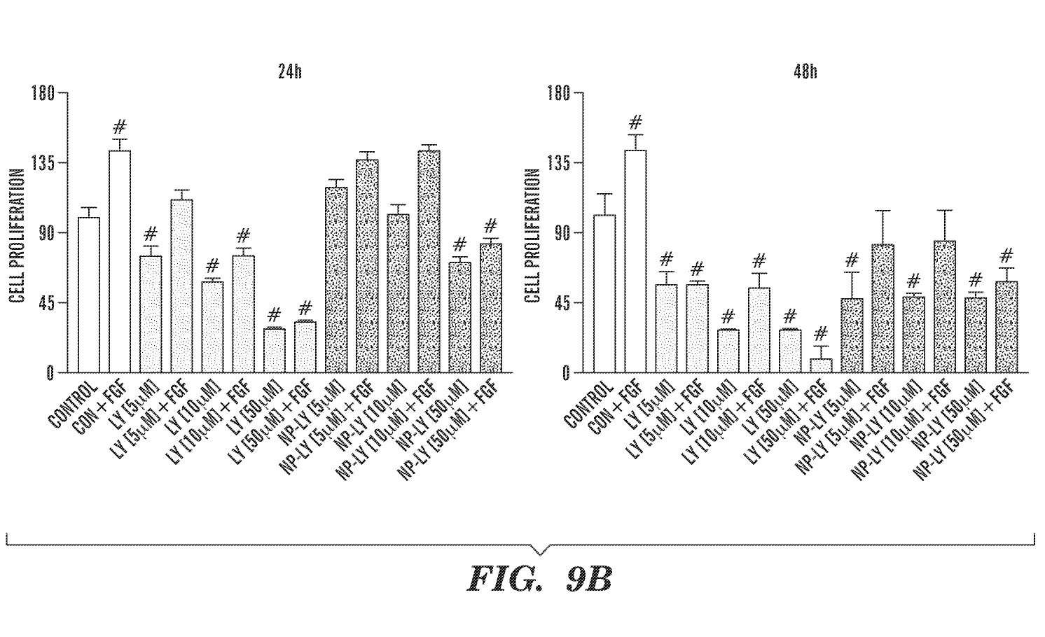

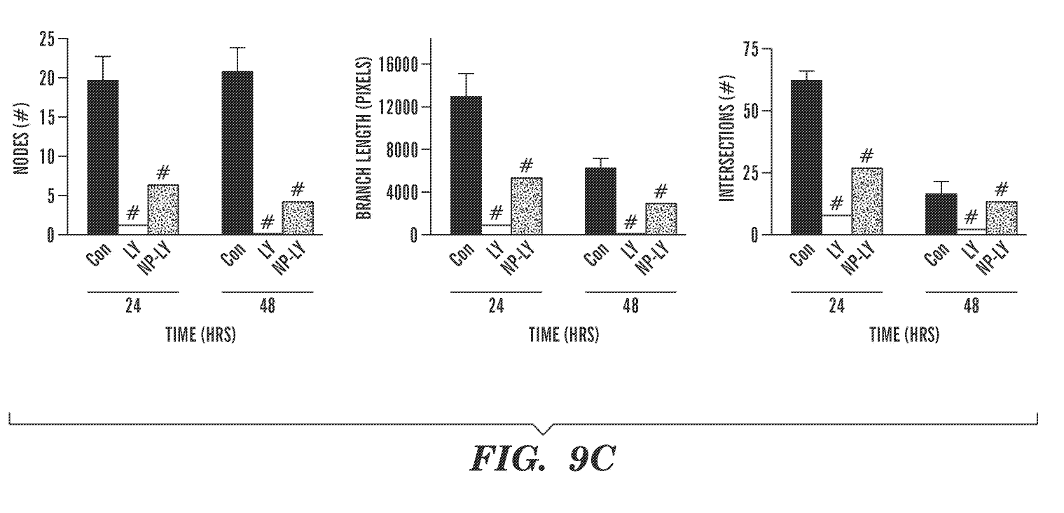

A key event during tumor progression is the requirement for angiogenesis, or the formation of new blood vessels from an existing vascular bed, for the tumor to grow beyond 1 mm³ in volume [28]. This ‘angiogenic switch’ has been implicated as a critical step for tumor progression and metastasis [29]. The genetic stability of endothelial cells means the absence of resistance development, and hence inhibition of tumor angiogenesis has evolved as an attractive therapeutic strategy for the management of tumors, with many candidates in clinics or clinical trials [30]. Interestingly, a critical promoter for tumor angiogenesis is the activation of the PI3K/AKT pathway. Without wishing to be bound by theory, the discrepancy in the susceptibility of different tumors to PI3K inhibitors could potentially be overcome by nanoparticle-mediated targeting of the activated PI3K/AKT signaling cascade in endothelial cells, given that the angiogenic process is consistent across different tumors. HUVECs were seeded on gelatin-coated glass coverslips in 24-well plates and incubated with FITC-labelled nanoparticles for various time-points, after which time they were stained with LysoTracker Red to label the lysosomes, fixed and subjected to fluorescence microscopy at 40× magnification. At least three independent measurements were performed per time-point. The inventors observed a rapid uptake of the FITC-labeled NP-LY into human umbilical vein endothelial cells (HUVECs) within 30 minutes of incubation, with internalization into the lysosomes clearly evident by 6 hours as seen from the colocalization of the signals from the FITC-NP and the lysotracker Red-labeled lysosomes (data not shown). Interestingly the FITC signal and the lysotracker signal disengaged by 12 hours, suggesting that the nanoparticles are processed in the lysosomes and the active agents are released into the cytosol (data not shown). Indeed, Western blot analysis of the activation of AKT revealed that 24 hours incubation with both LY294002 and NP-LY at 50 μM concentration resulted in complete inhibition of VEGF-induced activated PI3K-mediated phosphorylation of AKT in the HUVEC cells (FIG. 9A).

The angiogenesis process involves a temporal series of discrete but overlapping steps, including proliferation and tubulogenesis by endothelial cells [31]. The activity of the NP-LY on endothelial cell proliferation was evaluated. Serum-starved synchronized HUVECs were stimulated with fibroblast growth factor (FGF) in the presence of increasing concentrations of LY294002 or NP-LY. Cell proliferation at the end of 24 and 48 hours was quantified using an MTS assay. As shown in FIG. 9C, treatment with LY294002 or LY-NP blocked FGF-induced cell proliferation. Furthermore, at 24 hours, HUVEC proliferation was significantly inhibited only at the highest concentration of NP-LY, but by 48 h all three concentration of NP-LY had similar effect as the free drug. This is consistent with the temporal control over release exerted by encapsulating LY294002 in the nanoparticles. Interestingly, treatment with LY294002 or NP-LY failed to significantly reduce the cell numbers to below the basal level, suggesting that PI3K-blockade only inhibits the activated endothelial cell response, which could be critical in specific targeting of tumor vasculature that is activated unlike normal vessels.

During angiogenesis, proliferation of endothelial cells is followed by tubulogenesis, which is mimicked when endothelial cells are plated on growth factor-enriched Matrigel, a tumor extracellular matrix. As shown in FIG. 9C, HUVECs formed a well developed vascular network of tubes on Matrigel in the vehicle-treated group. In contrast, both LY294002 and NP-LY inhibited tubulogenesis as quantified by three different morphometric analysis (FIG. 9C). Interestingly, as confirmed by all three analyses, both free LY294002 and NP-LY significantly inhibited tube formation within 24 hrs; the free drug was more potent, again confirming the temporal nature of release of active drug from NP-LY. As such, 48 hrs of LY294002 or NP-LY treatment resulted in inhibition of branch length by 98% and 52%, the number of nodes by 99% and 88% and the number of intersections by 87% and 24%, respectively. The fact that the first two analysis methods most closely correlate with the acquired images suggests that measuring both tube length and the number of nodes provides a more valuable morphometric tool than simply using the graticule method that measures the number of intersections. These results indicated that PI3K plays a key role in critical steps of angiogenesis, and therefore could emerge as an exciting target for the inhibition of tumor angiogenesis, consistent with earlier observations [32].

To test whether the anti-angiogenic activity of NP-LY translates into an in vivo setting, a zebrafish tumor xenograft model was used. This model has evolved as a powerful model for studying angiogenesis given its ease of use, effectiveness and high-throughput [33,34]. Zebrafish [TubingenAB and tg(Fli:GFP)] embryos were maintained at 28° C. in standard E3 solution buffered with 2 mM HEPES. 48 hrs post-fertilization (hpf) embryos were anesthetized with 0.04 mg/ml of Tricaine. B16/F10 melanoma or MDA-MB231 cells were injected in the yolk sac space near the subintestinal vessels in anesthetized animals. Roughly 1000 tumor cells resuspended in matrigel were injected in each case, in the presence or absence of NP-LY. The total injected volume was maintained constant at 9.2 nL using a Nanoject II as reported earlier. [35]. To visualize the cells following injections, we labeled the B16-F10 cells with Qtracker-Red (Qdots) or used green fluorescent protein-stably transfected MDA-MB-231/GFP cells. Images were taken both in real-time and after staining zebrafish with nitroblue tetrazolium chloride and 5-Bromo-4-chloro-3-indolyl phosphate, toluidine salt in order to visualize their vasculature. Brightfield and fluorescence images of the embryos were captured with a Nikon SMZ1500 stereomicroscope and SPOT Flex camera. Image sequences were obtained with the same set-up and exported as movies to match live flow patterns.

The injected nanoparticles (FITC-labeled) were restricted in close proximity to the subintestinal vessels, and by themselves didn't induce cytotoxicity (data not shown). Similarly, the Qtracker-labelled B16/F10 cells were found to be viable and growing in the perivitelline space (data not shown), and in some cases showed metastasis. The tumor cells were found to induce angiogenesis as was seen from the remodeling or budding of vascular structures from the subintestinal vessels, consistent with previous observations [36]. The presence of NP-LY completely inhibited subintestinal vessel angiogenesis (data not shown). Interestingly, in the case of MDA-MB-231 breast cancer cells, we observed less metastasis than B16/F10 cells (data not shown) but significantly greater angiogenesis from the subintestinal vessels in the xenografted fish. Morphometric analysis revealed that the xenograft-induced angiogenesis was greater than 3× the angiogenesis observed in vehicle-controls (data not shown). Interestingly, the injection of NP-LY abrogated this vessel development to levels lower than those of control fish (data not shown). Treatment with NP-LY at a concentration range of 50 to 200 μM revealed a dose-dependent inhibition of subintestinal angiogenesis (data not shown), thus confirming an anti-angiogenic role of NP-LY in vivo. Interestingly, the consistency of the anti-angiogenesis response with NP-LY in the case of either tumor cells, despite their inherent distinctions in susceptibility to the treatment, indicates that a successful PI3K inhibitor-nanoparticle-based anti-tumor strategy should focus on inhibiting angiogenesis underlying tumor progression. Indeed an optimal strategy for effective anticancer outcome could combine such a signal-transduction inhibition-based anti-angiogenesis approach with a cytotoxic chemotherapy for optimal anticancer outcome, which we are currently exploring in our laboratory.

In summary, we have engineered nanoparticles that can inhibit the aberrant PI3K-AKT signaling pathway that is implicated in tumorigenesis. Interestingly, the inventors have discovered that different tumor cell lines show distinct susceptibility to a nanoparticle-based strategy for inhibiting the PI3K pathway, although angiogenesis induced by the cell lines is uniformly susceptible to the treatment. This discovery can be harnessed for nanoparticle-based inhibition of PI3K signaling for tumor anti-angiogenesis, which has evolved as an attractive strategy for the cancer therapy. Interestingly, it also highlights an important point that an anticancer approach need not only focus on the dividing cancer cells, but opportunities exist within the non-transformed component of the tumor, i.e. the stroma, which is comprised of vasculature and matrix. Indeed, nanoparticles targeted to αvβ3 integrins on tumor vasculature were found to ablate tumors in earlier studies [37]. Such a targeting mechanism is easily adapted to the LY-NPs described herein. Pegylated nanoparticles have been shown to preferentially home into tumors without any active targeting, arising from the passive uptake into the tumors because of the EPR effect. Therefore, the pegylated nanoparticles can enable preferential accumulation of the inhibitor in the tumors thereby increasing the therapeutic index. Using these pegylated nanoparticles, the clinical hurdles that have arisen from pharmaceutical challenges and off-target mechanism-driven toxicities associated with PI3K inhibitors can be easily overcome.

### REFERENCES

1. S. M. Moghimi, A. C. Hunter, and J. C. Murray. FASEB J. 2005; 19: 311
2. Yuan F, Leunig M, Huang S K, Berk D A, Papahadjopoulos D, Jain R K. Cancer Res. 1994; 54:3352
3. Ibrahim N K, Desai N, Legha S, Soon-Shiong P, Theriault R L, Rivera E, Esmaeli B, Ring S E, Bedikian A, Hortobagyi G N, Ellerhorst J A. Clin Cancer Res. 2002; 8:1038-44.
4. Stpyridi F, Palmieri C. Expert Rev Anticancer Ther. 2008; 8:1859-69.
5. Downward J. Targeting RAS signaling pathways in cancer therapy. Nature Rev. Cancer. 2003; 3: 11-22
6. Vivanco I, Sawyers C L. Nat Rev Cancer. 2002; 2:489-501.
7. Datta, S. R., Brunet, A. & Greenberg, M. E. Genes & Development 1999; 13, 2905-2927.
8. Shayesteh L, Lu Y, Kuo W L, Baldocchi R, Godfrey T, Collins C, Pinkel D, Powell B, Mills G B, Gray J W. Nat. Genet. 1999; 21:99-102.
9. Engelman J A, Chen L, Tan X, Crosby K, Guimaraes A R, Upadhyay R, Maira M, McNamara K, Perera S A, Song Y, Chirieac L R, Kaur R, Lightbown A, Simendinger J, Li T, Padera R F, García-Echeverria C, Weissleder R, Mahmood U, Cantley L C, Wong K K. Nat. Med. 2008; 14:1351-6.
10. Koul D. Cancer Biol Ther. 2008; 7:1321-5. 11.
11. Petrocelli T, Slingerland J M. Breast Cancer Res. 2001; 3:356-60.
12. Steelman L S, Stadelman K M, Chappell W H, Horn S, Bäsecke J, Cervello M, Nicoletti F, Libra M, Stivala F, Martelli A M, McCubrey J A. Expert Opin Ther Targets. 2008; 12:1139-65.
13. Iwanaga K, Yang Y, Raso M G, Ma L, Hanna A E, Thilaganathan N, Moghaddam S, Evans C M, Li H, Cai W W, Sato M, Minna J D, Wu H, Creighton C J, Demayo F J, Wistuba I I, Kurie J M. Cancer Res. 2008; 68:1119-27.
14. Watanabe S, Horie Y, Suzuki A. Hepatol Res. 2005; 33:161-6.
15. Yuan T L, Choi H S, Matsui A, Benes C, Lifshits E, Luo J, Frangioni J V, Cantley L C. Proc Natl Acad Sci USA. 2008; 105:9739-44.
16. Sengupta S, Sellers L A, Li R C, Gherardi E, Zhao G, Watson N, Sasisekharan R, Fan T P. Circulation. 2003; 107:2955-61.
17. Vlahos, C. J., Matter, W. F., Hui, K. Y. & Brown, R. F. J. Biol. Chem. 1994; 269, 5241-5248
18. Gaumet M, Vargas A, Gurny R, Delie F. Eur J Pharm Biopharm. 2008; 69:1-9.
19. Honorat, M. et al. Biochem. Biophys. Res. Commun. 2008; 375, 308-314
20. Stokoe D, Stephens L R, Copeland T, Gaffney P R, Reese C B, Painter G F, Holmes A B, McCormick F, Hawkins P T. Science. 1997; 277:567-70.
21. Datta S R, Dudek H, Tao X, Masters S, Fu H, Gotoh Y, Greenberg M E. Cell. 1997; 91:231-41.
22. Cardone M H, Roy N, Stennicke H R, Salvesen G S, Franke T F, Stanbridge E, Frisch S, Reed J C. Science. 1998; 282:1318-21.
23. Brunet A, Bonni A, Zigmond M J, Lin M Z, Juo P, Hu L S, Anderson M J, Arden K C, Blenis J, Greenberg M E. Cell. 1999; 96:857-68.
24. Buck E, Eyzaguirre A, Rosenfeld-Franklin M, Thomson S, Mulvihill M, Barr S, Brown E, O'Connor M, Yao Y, Pachter J, Miglarese M, Epstein D, Iwata K K, Haley J D, Gibson N R, Ji Q S. Cancer Res. 2008; 68:8322-32.
25. Grant S. Cotargeting survival signaling pathways in cancer. J Clin Invest. 2008; 118:3003-6.
26. She Q B, Solit D B, Ye Q, O'Reilly K E, Lobo J, Rosen N. Cancer Cell. 2005; 8:287-97.
27. Kinkade C W, Castillo-Martin M, Puzio-Kuter A, Yan J, Foster T H, Gao H, Sun Y, Ouyang X, Gerald W L, Cordon-Cardo C, Abate-Shen C. J Clin Invest. 2008; 118:3051-64
28. Folkman J. Angiogenesis: an organizing principle for drug discovery? Nat Rev Drug Discov. 2007; 6:273-86.
29. Folkman J. Role of angiogenesis in tumor growth and metastasis. Semin Oncol. 2002; 29:15-8.
30. Gasparini G, Longo R, Toi M, Ferrara N. Nat Clin Pract Oncol. 2005; 2:562-77
31. Sengupta S, Sellers L A, Cindrova T, Skepper J, Gherardi E, Sasisekharan R, Fan T P. Cancer Res. 2003; 63:8351-9.
32. Schnell C R, Stauffer F, Allegrini P R, O'Reilly T, McSheehy P M, Dartois C, Stumm M, Cozens R, Littlewood-Evans A, Garcia-Echeverria C, Maira S M. Cancer Res. 2008; 68:6598-607.
33. Stoletov, K., Montel, V., Lester, R. D., Gonias, S. L. & Klemke, R. Proc. Natl. Acad. Sci. U.S.A 2007; 104, 17406-17411.
34. Nicoli, S., Ribatti, D., Cotelli, F. & Presta, M. Cancer Res. 2007; 67: 2927-2931.
35. Nicoli, S. & Presta, M. Nat. Protoc. 2007; 2: 2918-2923.
36. Stoletov K, Montel V, Lester R D, Gonias S L, Klemke R. Proc Natl Acad Sci USA. 2007; 104(44):17406-11.
37. Hood J D, Bednarski M, Frausto R, Guccione S, Reisfeld R A, Xiang R, Cheresh D A. Science. 2002; 296:2404.
38. Sengupta S, Eavarone D, Capila I, Zhao G, Watson N, Kiziltepe T, Sasisekharan R. Nature. 2005; 436:568-72.
39. Kaul G, Amiji M. Biodistribution and targeting potential of poly(ethylene glycol)-modified gelatin nanoparticles in subcutaneous murine tumor model. J Drug Target. 2004; 12:585-91.
   All patents and publications cited herein are hereby incorporated by reference.

What is claimed is:

1. A modified polymer with increased drug-loading comprising:
a compound of formula (I): 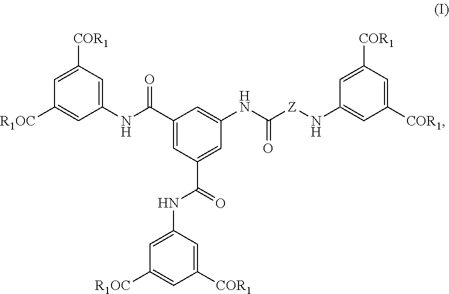
wherein Z is a poly(lactic-co-glycolic acid) (PLGA) having molecular weight from 1-15 kDa;
R₁ are independently H, R₂, OH, O-alkyl, —O—R₂, NH—R₂, -linker-R₂, or 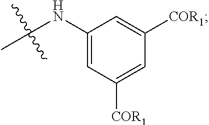
and
R₂ are independently one or more therapeutic agents.

2. The modified polymer of claim 1, wherein the PLGA polymer has a molecular weight from 3-8 kDa.

3. The modified polymer of claim 1, wherein the PLGA polymer has a molecular weight of 4 kDa.

4. The modified polymer of claim 1, wherein the PLGA polymer has a molecular weight of 7 kDa.

5. The modified polymer of claim 1, wherein PLGA polymer is represented by the formula (II): 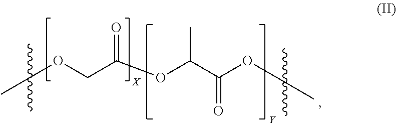
wherein the ratio of monomers X and Y ranges from 1:10 to 10:1.

6. The modified polymer of claim 5, wherein the ratio of monomers X and Y is from 25:75 to 75:25

7. The modified polymer of claim 5, wherein the ratio of monomers X and Y is 50:50.

8. The modified polymer of claim 1, wherein R₂ is a therapeutic agent with an amine group.

9. The modified polymer of claim 1, wherein said therapeutic agent is a kinase inhibitor.

10. The modified polymer of claim 9, wherein said kinase inhibitor is PD98059.

11. The modified polymer of claim 9, wherein said kinase inhibitor blocks one or more of VEGFR, PI3K, MET, EGFR, PDGFR, or erb2.

12. The modified polymer of claim 1, wherein said therapeutic agent is Lapatinib, Erlotinib, Vatalanib, Gefitinib, Nilotinib, Sunitinib, or TNP-470.

13. A nanoparticle drug delivery system comprising:
a PLGA-b-PEG block copolymer; and
a stabilizer.

14. The nanoparticle drug delivery system of claim 13 further comprising the modified polymer of claim 1.

15. The nanoparticle drug delivery system of claim 13 further comprising one or more additional therapeutic agents.

16. The nanoparticle drug delivery system of claim 15, wherein the additional therapeutic agent is at least one chemotherapeutic agent covalently bound to the PLGA.

17. The nanoparticle drug delivery system of claim 16, wherein the additional therapeutic agent is doxorubicin, a taxane, a podophyllotoxin, vinca alkaloids, or methotrexate.

18. The nanoparticle drug delivery system of claim 15, wherein the additional therapeutic agent is a PLGA-LY294002-PVA nanoparticle wherein the LY294002 is not covalently bound to the PLGA.

19. The nanoparticle drug delivery system of claim 13, wherein the stabilizer is polyvinyl alcohol (PVA).

20. A drug delivery system comprising:
PLGA-b-PEG block copolymer polyvinyl alcohol (PVA) nanoparticle; and the modified polymer of claim 1.
